(19) United States
(12) Patent Application Publication
(10) Pub. No.: US 2021/0255202 A1
(43) Pub. Date: Aug. 19, 2021

(54) METHODS AND DOSING REGIMENS FOR PREVENTING OR DELAYING ONSET OF ALZHEIMER'S DISEASE AND OTHER FORMS OF DEMENTIA AND MILD CONGNITIVE IMPAIRMENT

(71) Applicant: Michael R. D'Andrea, Mount Holly, NJ (US)

(72) Inventor: Michael R. D'Andrea, Mount Holly, NJ (US)

(21) Appl. No.: 16/973,586

(22) PCT Filed: Jun. 12, 2019
(86) PCT No.: PCT/US2019/036685

§ 371 Date: Dec. 9, 2020

Related U.S. Application Data
(60) Provisional application No. 62/684,454, filed on Jun. 13, 2018.

(51) Int. Cl. G01N 33/68; A61K 45/06; A61P 25/28

(52) U.S. Cl. CPC G01N 33/6896; A61K 45/06; G01N 2800/2821; G01N 2333/4709; A61P 25/28

(57) ABSTRACT

Methods and dosing regimens using alpha 7 acetylcholine nicotine receptor binding agents are provided to prevent or inhibit intracellular accumulation of amyloid in cells leading to inhibition or prevention of neuronal cell death. In addition, these methods and dosing regimens are coupled with methods and dosing regimens to reduce and/or prevent blood-brain barrier leakage of vascular-derived amyloid into the brain and/or methods and dosing regimens to reduce and/or prevent neuroinflammation to prevent and/or inhibit the progression of Alzheimer's disease and other forms of dementia and mild cognitive impairment. Also provided are methods for identifying individuals for this treatment.

This patent application claims the benefit of priority from U.S. Provisional Application Ser. No. 62/684,454 filed Jun. 13, 2018, teachings of which are herein incorporated by reference in their entirety.

### FIELD

The present invention relates to a 3-pronged therapeutic approach to prevent or delay onset and/or progression of Alzheimer's disease and other forms of dementia, and mild cognitive impairment (MCI). Methods and dosing regimens described herein involve the use of alpha 7 acetylcholine nicotinic receptor (A7R) binding agents to prevent and/or inhibit intracellular accumulation of amyloid in cells leading to inhibition or prevention of neuronal cell death, memory/learning impairment and/or Alzheimer's disease and other forms of dementia, and MCI. Methods and dosing regiments may further involve preventing unregulated entry of vascular-derived amyloid through a dysfunctional blood-brain barrier (BBB) into the brain, and/or reducing neuroinflammation. In addition, methods for identifying individuals for this therapeutic treatment are described.

### BACKGROUND

Diagnoses of Alzheimer's disease, the most common type of dementia that generally describes loss of memory and other mental abilities severe enough to interfere with daily life, are increasing at an alarming rate. Today, as many as half of the population over 80 years of age will be afflicted [76]. Alzheimer's disease is officially the sixth leading cause of death in the United States and fifth leading cause of death for those of ages 65 and older; far more than prostate cancer and breast cancer combined [65]. Further, deaths from Alzheimer's disease increased 68% between years 2000 and 2010, and Alzheimer's disease is among the top 10 causes of death in America that cannot be prevented, cured, or even slowed down [65]. It is estimated that 13.8 million Americans will be living with Alzheimer's disease by year 2050, up from 4.7 million in year 2010, and according to the World Health Organization, about 35.6 million people around the world have dementia, with 7.7 million new cases each year [101].

This disease not only negatively impacts the immediate family and friends of the victim but also is one of the most costly modern medical conditions to support. In year 2014, the direct costs to the American society for Alzheimer's disease care was estimated to be $214 billion. If there is no breakthrough cure or way to prevent or even slow down the progression of Alzheimer's disease, the costs could reach up to $1.2 trillion by year 2050 [65].

The most widely accepted hypothesis explaining the cause of Alzheimer's disease is referred to as the amyloid cascade hypothesis, and is generally based on neurons over-producing and secreting toxic amyloid that is deposited between neurons in the extracellular spaces of the brain where it eventually kills neighboring neurons. The following 2017 statement embodies the orthodoxy that governs the essence of the Alzheimer's disease field. “Alzheimer's disease results from progressive brain degeneration due to the formation of harmful plaques and neurofibrillary tangles. These protein abnormalities block neuron connections, eventually leading to neuron death and brain tissue loss. Ultimately, long-term brain deterioration stimulates dementia onset, which involves symptoms such as memory loss, personality changes, problems with language, and confusion. This debilitating condition increases in severity over time and, as it has no cure, people with Alzheimer's disease often require constant care” [63]. Simply stated, over time, the amyloid grows in size, shape, and form to become more fibular and toxic leading to the destruction of neighboring neurons. These areas of extracellular amyloid are commonly referred to as amyloid plaques and are the basis of the neuropathology in the Alzheimer's disease brain. Therefore, the targets to cure Alzheimer's disease are first, to prevent the accumulation of the toxic form of amyloid, referred to amyloid beta (Abeta)42, from production through inhibitors and second, to prevent the amyloid from growing or maturing (i.e., monomer to polymer to fibrils) in the areas of the brain between neurons.

The amyloid cascade hypothesis has been the cornerstone of Alzheimer's disease research for decades. This hypothesis further states that extracellular amyloid deposits, generated by the proteolytic cleavages of amyloid precursor protein (APP), are the fundamental cause of Alzheimer's disease. As a result of its widespread acceptance, hundreds of publications focus on understanding the processing pathway of APP, Abeta production and its enzymatic partners (beta- and gamma-secretase, beta-secretase, etc.), the function and properties of its cleaved products (Abeta40, Abeta42, etc.), and how they relate to Alzheimer's disease. Although Abeta40 and Abeta42 have been reported in plaques, the Abeta42 form is more directly toxic, has a greater propensity to aggregate, and is the most studied form of amyloid. Under normal conditions, about 90% of secreted Abeta peptides are Abeta40, which is a soluble form of the peptide that only slowly converts to an insoluble beta-sheet configuration and, thus, can be eliminated from the brain. In contrast, about 10% of secreted Abeta peptides are Abeta42, which is the species that is highly fibrillogenic and deposited early in individuals with Alzheimer's disease and Down syndrome subjects. Intracellular assembly states of Abeta are monomers, oligomers, protofibrils, and fibrils. The monomeric species are not pathological, although the nucleation-dependent fibril formation related to protein misfolding makes the Abeta toxic. The oligomeric and protofibrillar species may facilitate tau hyperphosphorylation, disruption of proteasomal and mitochondrial function, dysregulation of calcium homeostasis, synaptic failure, and cognitive dysfunction. This hypothesis is further supported by the fact that all Down syndrome subjects, who have the extra 21 chromosome that contains the APP gene, will have Alzheimer's disease by the age of 40.

In addition, apolipoprotein E (ApoE; discussed in detail below) mediates Abeta metabolism, where it can bind to Abeta to affect its deposition and clearance, and is required for amyloid deposition in an allele-specific manner. Preclinical transgenic mice that express a mutant form of the human APP gene develop fibrillar amyloid plaques and Alzheimer's disease-like pathology with spatial learning deficits. These extracellular amyloid deposits or plaques grow in size and become more toxic, eventually killing neighboring neurons and leading to Alzheimer's disease.

The belief within the Alzheimer's disease community in this hypothesis is clearly evidenced by the funding of several highly publicized clinical trials. In many ways, the fate of Alzheimer's disease research is contingent on the accuracy of this hypothesis and the success of the clinical trials meant to test the hypothesis.

In the United States alone, government initiatives have funded $2.5 billion in Alzheimer's disease research just over the past 4 years including a projected $566 million in 2015 [84]. Despite these impressive funding numbers, researchers dedicated to the field produced very few breakthroughs. Generally, many scientists believe they can cure Alzheimer's disease by removing the amyloid between the neurons in the brain before they kill neighboring neurons as per the amyloid hypothesis. However, a majority of these trials have failed to deliver, for seemingly unresolved reasons. The last few years have been discouraging for the estimated 50 million dementia sufferers worldwide who are waiting for the first treatments that can arrest the devastating brain diseases [101]. Drug after drug has failed the final stage of testing, even after earlier clinical trials offered hope that the experimental medicines might be able to slow the relentless march of the illness. The failing streak of clinical trials continued through today (as of June, 2019).

For example, since early 2018, headlines read: “Pfizer halted their research after dismal results from their Alzheimer's disease trials” [82], “Axovant abandoned their banner Alzheimer's disease drug Intepridine” [83], “Merck discontinued their APECS study for the treatment of prodromal Alzheimer's disease” [70], and “Eli Lilly's antibody Solanezumab failed to reach statistical significance in their Expedition Alzheimer's disease trials” [66,67]. And early 2019, “Biogen ended 2 Alzheimer's disease trials with Aducanumab” [98].

Specifically, in most of these recent efforts to cure Alzheimer's disease subjects were treated with an anti-amyloid antibody to remove the amyloid in the brain with the hope to improve memory, lucidity, and other clinical maladies. The antibody, Bapineuzumab, was then tested in several clinical trials. However, the drug failed to achieve the desired end points. Further, pharmaceutical companies involved this particular trial announced that their Alzheimer's drug had yielded such bad results that they were stopping all further work, “dashing hopes for the 5 million Americans suffering from Alzheimer's disease and becoming the latest piece of evidence of the drug industry's strange gambling problem” with very high investments all into one endeavor [77]. Aducanumab, another anti-amyloid antibody, was designed by Biogen to clear the brain of sticky plaques known as “beta-amyloid”, which accumulate in the brains of people with Alzheimer's, and which some scientists blame for the disease. Although Biogen's drug appeared to be able to remove those plaques, efficacy endpoints were also not met [78, 98]. In another Phase 3 trial, Solanezumab, yet another anti-amyloid antibody, was given to individuals with mild Alzheimer's disease, unfortunately, the study failed to reach statistical significance as well.

A large Phase 3 study evaluating Verubecestat (MK-8931), an investigational small molecule inhibitor of the beta-site APP cleaving enzyme 1 (BACE1), in people with prodromal Alzheimer's disease was recently stopped based upon an overall benefit/risk assessment during a recent interim safety analysis [70,71]. Other similar clinical studies using beta-secretase inhibitors R G7129 and LY2886721 on subjects that held the promise of preventing the production of amyloid beta were also recently stopped due at least in part to toxicity [72].

Nonetheless, clinical trials are still ongoing for a potential drug being developed by Amgen/Novartis for people with no outward signs of Alzheimer's disease, but who carry a gene that makes them more predisposed to developing the disease in the future [78]. This potential drug also attempts to inhibit the enzyme known as beta-secretase, which is implicated in the formation of amyloid plaques.

Intepirdine is a non-amyloid-based experimental Alzheimer's drug by Axovant Sciences, Inc. that blocks the 5HT6 receptor from promoting the release of acetylcholine within the brain. Aricept, a cholinesterase inhibitor, also increases acetylcholine, but in a nonselective and indirect way, by preventing its breakdown. When used together with Aricept, they increase the concentration of acetylcholine through a complementary mechanism without worsening Aricept's side effects, such as nausea and vomiting [64]. However, Intepirdine also recently failed to meet the goals of a pivotal trial [99, 102].

Vascular risk factors are associated with the development of Alzheimer's disease. The vascular hypothesis suggests that the pathology of Alzheimer's disease begins with a decreased blood flow or hypo-perfusion to the brain. Support for a vascular cause of Alzheimer's disease comes from epidemiological, neuroimaging, pathological, and clinical trials [37,136,163]. This hypothesis considers cerebral microvascular pathology and cerebral hypo-perfusion as primary triggers for neuronal dysfunction leading to the cognitive and degenerative changes in Alzheimer's disease [124]. Advancing age and the presence of vascular risk factors create a critically attained threshold of cerebral hypo-perfusion that ultimately leads to capillary degeneration [133]. Thus, the pathological consequences of capillary degeneration result in the development of amyloid plaques, inflammatory responses, and synaptic damage, which leads to the manifestations of Alzheimer's disease [36].

Therefore, vascular targets have been considered to cure Alzheimer's disease. For example, significant evidence linked high levels of cholesterol to Alzheimer's disease, and several clinical trials showed a reduced risk for Alzheimer's disease in populations treated with statins, which are drugs made to lower cholesterol levels. Maintaining normal levels of cholesterol is essential for the prevention of disorders of the cardiovascular system, including hypertension, heart attack, stroke, and hypercholesterolemia; all of which are Alzheimer's disease risk factors [112]. The role of cholesterol in the pathology of Alzheimer's disease is also shown by the ability of statins to reduce the prevalence of Alzheimer's disease by up to 70%. Intracellular cholesterol regulates amyloid processing by directly modulating the activity of secretase, which is the enzyme that breaks down the amyloid protein into smaller parts. Cholesterol also affects the intracellular trafficking of amyloid and secretase [58]. In particular, high intracellular cholesterol increases gamma-secretase activity and amyloidogenic pathways, while low intracellular cholesterol favors non-amyloidogenic pathways. Inhibition of cholesterol biosynthesis by statins and another cholesterol synthesis inhibitor were found to reduce amyloid burden in guinea pigs and murine models of Alzheimer's disease [133]. A substantial body of cellular and molecular mechanistic evidence links cholesterol and Abeta generation to Alzheimer's disease [38,106,109,148,149,151,161,164,187] and has helped support clinical trials of statins in persons with Alzheimer's disease. Such clinical trials reported reduced risk for incidence and progression of Alzheimer's disease in statin-treated populations [58, 108,151,183]. However, this approach has failed in a randomized, controlled clinical trial, where a 72-week course of treatment of Atorvastatin was given to 640 subjects with mild to moderate Alzheimer's disease; the subjects did not improve cognitive measures [50]. In a 18-month, randomized, placebo-controlled trial, Simvastatin,a natural statin derived from fermentation, was given to 406 subjects with mild to moderate Alzheimer's disease; the subjects also did not show advantageous clinical effects [156]. Furthermore, in another placebo-controlled Simvastatin trial, Simvastatin did not significantly alter cerebrospinal fluid levels of Abeta; although, there was evidence for efficacy in Abetal-40 reduction in persons with “mild” Alzheimer's disease [162]. Therefore, clinical trials evaluating statins in general lzheimer's disease populations were unable to show significant therapeutic benefit [8,50, 53,131,156,162].

Genetics has also been implicated in the development of Alzheimer's disease. Those who have a parent or sibling with Alzheimer's disease are more likely to develop the disease and this probability continues to increase if more than one relative have or had Alzheimer's disease. Although this suggests that Alzheimer's disease has a significant genetic component, the known genetic risks account for only 0.1% of Alzheimer's disease cases. The most prominent genetic risk factor is the gene that codes for apolipoprotein E (APOE4) [185]. The APOE2 and APOE3 gene forms are the most common in the general population, but it is the APOE4 gene that is associated with an individual's risk for developing late-onset Alzheimer's disease. These lipoproteins are responsible for packaging cholesterol and other fats, and for transporting them through the bloodstream. ApoE is also a major component of a specific type of lipoprotein, known as very-low-density lipoproteins, which remove excess cholesterol from the blood to the liver for processing. ApoE also has a role in neuronal signaling and the maintenance of the integrity of the BBB that regulates the entry of selective substances into the brain. However, the exact pathophysiological process is yet to be elucidated. Although APOE is the only gene with replicable evidence, several candidate genes involved in lipid metabolism are being investigated for putative roles with mixed results [177].

Targeting neurons is another area of development to cure Alzheimer's disease. One of the major discoveries in the 1970s was a deficit in choline acetyltransferase, an enzyme that synthesizes the neuronal transmitter acetylcholine, in the neocortex of the Alzheimer's disease brain. Studies reported reduced choline uptake, increased acetylcholine release, and the degeneration of cholinergic neurons (those that use acetylcholine as a neurotransmitter) in specific areas in the Alzheimer's disease brain [12,35,141,146,155,181]. These data make up the foundation of the cholinergic hypothesis which suggests that the loss of cholinergic neurons, and thus the loss of cholinergic neurotransmission in critical brain areas, contributes significantly to the deterioration, in cognitive function of Alzheimer's disease subjects [7]. The contemporary discovery of acetylcholine's pivotal role in learning and memory further supports this hypothesis [42]. Today, the cholinergic hypothesis is the basis of most of the currently available drug therapies to treat Alzheimer's disease, which are meant to inhibit cholinesterase, an enzyme that breaks down acetylcholine. Unfortunately, as of today, these therapies have had minimal success in curing Alzheimer's disease [51].

Another neuronal target presented to cure Alzheimer's disease is a specific neuronal receptor named the alpha-7 nicotinic acetylcholine receptor (A7R). This receptor consists of homomeric A7 subunits, and is a ligand-gated Ca²-permeable ion channel implicated in cognition, learning, mood, emotion, neuroprotection, and neuropsychiatric disorders. Enhancement of A7R function is considered to be a potential therapeutic strategy aiming at ameliorating cognitive deficits of neuropsychiatric disorders such as Alzheimer's disease and schizophrenia. The functions of A7Rs are critical for cognition, sensory processing, attention, working memory, and reward. On the contrary, dysfunctional A7Rs are associated with multiple psychiatric and neurologic diseases including schizophrenia, Alzheimer's disease, attention deficit hyperactivity disorder, addiction, pain, and Parkinson's disease. Thus, modulation of A7R function is an attractive strategy for potential therapy of CNS (central nervous system) diseases. Currently, a number of A7R modulators have been reported and several of them have advanced into clinical trials. As reviewed in Yang et al, 2017 [188], there are 11 drug candidates of which 10 agonists and 1 positive allosteric modulator are currently being tested for treatment of schizophrenia, 9 agonists for Alzheimer's disease, 3 agonists for nicotinic addiction, 2 agonists for attention deficit hyperactivity disorder, and 1 agonist each for Parkinson's disease and pain. Unfortunately, most of the clinical trials using A7R agonists have been terminated or suspended (see Table 1 in Yang et al, 2017 [188]).

Another prominent hypothesis presented to cure Alzheimer's disease was presented in the 1980s and named the inflammation hypothesis, whereby neuroinflammation was identified as the cause of neuronal death in the Alzheimer's disease brain. In fact, the discovery of a wide array of immune-related antigens in the Alzheimer's disease brain helped establish the concept of a specialized immunodefense system in the CNS. In particular, as a result of some factors in the Alzheimer's disease brain, microglia become reactive and set off a chain of events releasing immune-related antigens including proinflammatory cytokines and chemokines [135]. According to the inflammation hypothesis, the increased secretion of these potentially neurotoxic substances eventually destroys neurons, leading to the development of Alzheimer's disease symptoms [115]. Some proponents of the inflammation hypothesis also suggest that this sequence triggers the distortion of tau via phosphorylation [115]. Even today, the role of inflammation in Alzheimer's disease is still widely debated.

However, evidence from numerous epidemiological studies show that Alzheimer's disease can be prevented by blocking inflammatory reactions with nonsteroidal inflammatory drugs (NS AIDs) that develop during the course of the disease [46,48,119,157,176]. NS AIDs are a category of medications that include the salicylate, propionic acid, acetic acid, fenamate, oxicam, and the cyclooxygenase (COX)-2 inhibitor classes [168]. Over 20 epidemiological clinical trials determined that anti-inflammatory drugs like Indomethacin and Ibuprofen reduce the risk of Alzheimer's disease [127,128,129]. Similarly, a decreased risk of Alzheimer's disease was observed in subjects with rheumatoid arthritis and osteoarthritis who were treated with NS AIDs for long periods of time [119]. Although clinical trials appeared to show that NS AIDs can prevent the risk of Alzheimer's disease, the results of clinical trials with anti-inflammatory drugs in Alzheimer's disease subjects were negative; especially for the COX-2 inhibitors [2,3,4,57,104,166].

Despite the number and range of attempts to understand the histopathology of Alzheimer's disease, how the neurons die, and how to treat Alzheimer's disease, the current therapies can only help treat the symptoms; there is no available treatment to stop or reverse the progression of Alzheimer's disease. The first line of treatment for Alzheimer's disease after diagnosis is cholinesterase inhibitor therapy. It is because the levels of acetylcholine are significantly reduced in subjects with Alzheimer's disease, that cholinesterase therapy with Rivastigmine, Donepezil, or Galantamine is administered to inhibit the actions of its natural degrading enzyme [49]. Subsequent treatment options for subjects with moderate to severe Alzheimer's disease include a combination therapy with the acetylcholinesterase inhibitor and Memantine (Namenda) [182].

There is a need for therapeutic interventions to do more than merely manage or treat the symptoms without targeting the cause or causes of Alzheimer's disease.

### SUMMARY

An aspect of the present invention relates to a method for binding or reducing toxic accumulation of amyloid in cells via administering a specific A7R binding agent to the cells.

Another aspect of the present invention relates to a method for preventing, inhibiting, and/or delaying the onset of Alzheimer's disease and other forms of dementia and MCI by administering to a subject one or more specific A7R binding agents. In one nonlimiting embodiment, the method further comprises administering one or more agents to reduce neuroinflammation and/or one or more agents to remedy BBB dysfunction.

In one nonlimiting embodiment, the A7R binding agent is administered to a subject prior to the onset of Alzheimer's disease.

In one nonlimiting embodiment, the A7R binding agent is administered to a subject at risk for developing Alzheimer's disease, other dementias and/or MCI.

Another aspect of the present invention is related to combination therapies to prevent, inhibit, and/or delay the onset of Alzheimer's disease and other dementias and MCI which comprise one or more A7R binding agents, one or more agents to reduce neuroinflammation, and one or more agents to remedy BBB dysfunction.

Yet another aspect of the present invention relates to a method for identifying an individual at risk for developing Alzheimer's disease and other dementias and MCI via assessment of blood-retina barrier (BRB) health and other biomarkers.

### DETAILED DESCRIPTION

The invention is based on a uniquely defined pathological pathway leading to the onset of Alzheimer's disease (and possibly other dementias and MCI) that begins with a dysfunction in the BBB. The loss of BBB regulation to control what can and cannot enter the brain leads to the unregulated pouring in of vascular components into the brain such as amyloid and immunoglobulins [23]. Like CNS-neuronal-produced amyloid, vascular-derived amyloid also binds to high-affinity A7Rs on neurons (as well as A7R-positive smooth muscle and endothelial cells) that internalize the amyloid. Over time, lethal amounts enter the cells leading to their death. The lysis of the amyloid-laden neurons leads to a cascade of secondary consequences of additional neuronal deaths. Initially, other nearby neurons die from trauma by the released enzymes from the lysed neurons which injure their local neuronal processes leading to degeneration. Subsequently, the products of the lysed neurons trigger neuroinflammation or gliosis by the activated microglia and reactive astrocytes, which then secrete factors that lead to the degeneration of neighboring neurons. At first, these events lead to MCI that over time lead to the onset of Alzheimer's disease, and other dementias [23,103].

Amyloid plaques, the hallmark of Alzheimer's disease histopathology, have been mostly described by their morphology without regard to etiology. Generally, there are the diffuse and dense-core amyloid plaques, and are believed to form from neuronally-produced amyloid detected in the extracellular synaptic spaces that initially appear diffuse and then over time, mature into the dense-core plaques. However, they are unique plaque types with distinct etiologies whereby the diffuse form from leaky vessels and therefore, this amyloid is vascular-derived, while the dense-core form as vascular-derived, Abeta-overburdened neurons die leaving their neuronal debris in place [28]. Imaging by the inventor showed the diffuse-type Abeta42 plaques in the precise shape of the longitudinal sectioned vessel of Alzheimer's disease serial cortical sections; hence, these diffuse amyloid plaques are not randomly located, and therefore, can be characterized as extracellular vascular-associated plaques. However, no Abeta42 was detected around the nearby veins suggesting an arterial source of the amyloid that is further observed with the presence of Abeta42 in the vascular arterial smooth muscle cells. Although it is believed that extracellular Abeta42 is toxic, its presence did not disrupt proteolytically-sensitive microtubule-associated protein-2 (MAP-2) labeling patterns like that of the dense-core type of amyloid plaque, which is discussed in detail below [29]. Furthermore, no inflammatory cells were associated with these diffuse amyloid plaque types using a novel triple-immunohistochemical immunolabeling method was designed by the inventor to simultaneously identify amyloid, activated microglia, and reactive astrocytes (further discussed below) [24]. To further provide evidence that the vessels and BBB are dysfunctional allowing unregulated vascular components into the brain, control and Alzheimer's disease brains were processed for immunohistochemistry to identify immunoglobulins. Results show the presence of significantly more vascular-derived IgGs in the Alzheimer's disease brains than in age-matched control brains [25,26]. To verify these interpretations in mice, an experiment was performed whereby mice in the treated group were injected (tail vein) with pertussis toxin, a bacterium known to cause BBB leakage [116], and FITC-labeled Abeta42. Diffuse, FITC-labeled Abeta42 was detected around vessels that were not detected in the untreated mice [16].

These data are supportive of that vascular-derived amyloid (and other vascular components such as IgGs) can enter the brain through a dysfunctional BBB in the vessels of the brain and leak into the brain parenchyma forming these benign, diffuse, vascular-associated amyloid plaques without triggering gliosis.

By “a” or “an” when used herein with respect to a therapeutic agent, it is meant to include use of one or more of those therapeutic agents.

The present invention provides methods and dosing regimens to treat BBB dysfunction. Overwhelming evidence shows that vascular pathologies are not only present in Alzheimer's disease but may actually be one of the earliest pathological events leading to the disease. It is not clear which groups of subjects with vascular diseases eventually develop Alzheimer's disease; however, it is clear that vascular pathology is a prerequisite for Alzheimer's disease. All of the cells of the vascular system contribute to this pathology, which appears to begin from intracellular Abeta in smooth muscle cells, as well as in endothelial cells [33]. Although the source(s) of the Abeta seems to come from the vascular system, the presence of Abeta receptors provides a mechanism of endocytosis for the intracellular Abeta (discussed further below). The collective orchestration of the vascular cells helps to maintain the barrier function, and it is clear that all of these cells are negatively impacted by intracellular levels of Abeta. Loss of BBB function, which is present in most of the Alzheimer's disease brains, has been found by the inventor to lead to focal areas of vascular leakage. It was reported that leakage of the BBB is associated with other neurological disorders, including temporal lobe epilepsy [169]. Following ischemic stroke, the integrity of the BBB can be impaired in cerebral areas distant from the initial ischemic insult, a condition known as diaschisis, leading to chronic poststroke deficits [52]. In the ischemic rat brain model, the late administration of vascular endothelial growth factor (VEGF) enhanced angiogenesis in the ischemic brain, improved neurological recovery, and the early administration of VEGF exacerbated BBB leakage. Hence, the controlled regulation of VEGF could be a potentially effective therapeutic strategy aimed at administration of exogenous VEGF to promote therapeutic angiogenesis during the repair process after a stroke and inhibition of VEGF at the acute stage of stroke to reduce the BBB permeability and the risk of hemorrhagic transformation after cerebral ischemia [190].

In the present invention, therapeutics directed to treat or prevent vascular disorders associated with diabetes and hypercholesterolemia could also be effective as the treatment of preclinical models of these pathological conditions with Darapladib, a selective inhibitor of lipoprotein-associated phospholipase-A2 which blocked the progression of atherosclerosis while reducing BBB leakage [1]. Statins ameliorate BBB dysfunction resulting from a number of conditions, including diabetes, transient focal cerebral ischemia, and HIV-1 [118,134,137]. The treatment of Simvastatin was effective in reducing the BBB permeability as measured by Evan's blue dye across the BBB in rabbits fed a cholesterol-enriched diet [106].

Clinical trials to cure Alzheimer's disease using compounds to treat the vascular system have failed (as described in the background section) because the neurons that cause the onset of Alzheimer's are already dead and therefore, treating the vascular system is too late and will never resurrect dead neurons as per the pathological mechanism described in this invention. Therefore, cognitive improvement efficacy endpoints are unrealistic. The true efficacy endpoint should be to merely prevent vascular leakage, but well before the diagnosis of Alzheimer's disease, preferably before or just after the diagnosis of MCI or sooner if predictive test become available. However, based on the novel mechanism present in this invention, even if the BBB is therapeutically resolved, neurons will continue to die since Abeta-laden neurons are already in the process of degenerating (i.e., generate the clinical symptoms), and other neurons will continue to die independent of amyloid due to the deleterious toxic effects of gliosis. Although clinical trials treating the vascular system have failed, such compounds that prevent BBB leakage are expected to be useful in inhibiting or preventing Alzheimer's disease and other dementias and MCI when used according to this invention in a prophylactic therapeutic approach and in combination in accordance with the present invention with other therapeutic agents which block over-accumulation of Abeta into neurons via A7R and which block neuroinflammation. Once the diagnosis of MCI is made (or sooner), there is a therapeutic window of opportunity to intervene to 1) stop further pouring of amyloid from the vascular system into the brain, 2) prevent further intraneuronal accumulation of amyloid before they lysis, and 3) suppress the ongoing processes of gliosis.

Results disclosed herein are indicative of Abeta42-positive dense-core amyloid plaques originating from the lysis of individual, Abeta42-burdened neurons. To begin, intraneuronal Abeta42 is detected in age-matched, non-demented brains suggesting that Abeta42 is hardly toxic [28]. Other than the occasionally observed diffuse, vascular-associated Abeta plaques, infrequent neurons do show the presence of excessive intracellular loading of Abeta42. Conversely, in Alzheimer's disease brains, the amounts of intracellular Abeta42 in significant numbers of neurons increase to the point of inflicting degeneration (e.g., condensed, pkynotic nuclei), some of which appear to have burst forming the dense-core plaque, initially suggesting that over time, neurons lyse due to over-accumulation of Abeta42 leaving a residual hematoxylin-stained blue nuclei.

Supportive evidence comes from a study of lipofuscin, that is often used as a histological index of aging, and originates from lysosomes. Lipofuscin is a special category of heavily oxidized, indigestible material that gradually accumulates in long-lived cells such as neurons [189]. Increases in lipofuscin above normal levels in neurons have been reported to be associated with neurodegenerative diseases including Alzheimer's disease [39,42,43,44,120]. If Abeta42 and lipofuscin are co-localized, it is conceivable that the observed increases in neuronal lipofuscin associated with Alzheimer's disease may actually be facilitated by intracellular accumulation of Abeta42-positive material and its deposition within the same intracellular compartment. To examine this possibility, a combined IHC:histochemical staining protocol is designed to simultaneously localize lipofuscin and Abeta42 in the tissue sections [31]. Although there is some detectable co-localization, most of the lipofuscin was purely restricted to the neuronal perikaryon, while the Abeta42 was located in the neuronal dendritic processes as well as in the perikaryon; therefore they occupy distinct cellular compartments in neurons of normal, age-matched control and Alzheimer's disease tissues [30]. The labeling patterns of the lipofuscin also show that most of this material is not co-localized with Abeta42 in neurons or in amyloid plaques. In addition, yellow-pigmented, unstained, lipofuscin has been located towards the center of some of the dense-core type, amyloid plaques.

Further evidence for the neuronal origin of dense-core amyloid plaques shows that nuclear material is also located in the middle of these types of plaques. For example, the presence of NeuN, a neuronal-specific nuclear protein, is also located in the center of some of the Abeta42-positive dense-core plaques through double immunohistochemical methods, and therefore confirms the presence of a neuronal nucleus at the center of this amyloid plaque type. In addition, neuronal-specific mRNAs have also been detected in these type of plaques [54]. Centromeric DNA repeat sequences were detected dispersed throughout areas of FITC-labeled, Abeta42-positive dense-core plaques using fluorescent in situ hybridization. In fact, some dense-core amyloid plaques have an intact, DAPI-specific nuclear remnant positioned at or near the amyloid plaque dense-core, which have similar morphology to the hematoxylin-stained nuclei seen in neurons with excessive Abeta42 accumulation. Also, treatment of the same DAPI-stained slides with DNase I abolished the DAPI-positive DNA stain within the dense cores confirming the specificity of the DAPI DNA stain [32].

In addition to the nuclear evidence (e.g., NeuN, mRNA, DNA), cytoplasmic neuronal proteins such as neurofilament proteins, tau, ubiquitin, and cathepsin D [28,32] are also detected in dense-core plaques suggesting these materials must be resilient enough to remain in place after the neuron dies or lyses. Therefore, if the detectable material that remains in the wake of the dead neuron is proteolytically-resistant to the release enzymes as the neurons die or lyse, then the opposite should be true that proteolytically-sensitive neuronal proteins would be absent, or not detectable due to their digestion. To further test the lysis hypothesis, the distribution of MAP-2, a protein localized primarily in neuronal dendrites and known to be sensitive to proteolysis, is examined in Alzheimer's disease brains [29]. Uniform MAP-2 immunolabeling is detected throughout the somatodendritic compartment of neurons in age-matched control cortical brain tissues as well as throughout areas of Abeta42-positive diffuse plaques in Alzheimer's disease brains using double immunohistochemical methods. In contrast, analysis of serial sections as well as double immunohistochemical stained slides to simultaneously show MAP-2 and Abeta42, methods reveal that MAP-2 is absent precisely in the areas of the Abeta42-positive dense-core plaques in Alzheimer's disease brains [29]. These results further indicate that this differential MAP-2 immunolabeling pattern could be employed as a reliable and sensitive method to distinguish dense-core plaques from diffuse plaques within Alzheimer's disease brain tissue. Furthermore, this biochemical distinction indicates that dense-core and diffuse plaques are formed through unique mechanisms and that digested MAP-2 could serve as a biomarker of neuronal death if detected in bodily fluids (e.g., cerebral spinal fluid, blood) (further discussed below).

Additional evidence for the lysis hypothesis that dense-core amyloid plaques originate from lysed Abeta42-overburdened neurons is supported by the lack of these types of plaques in the molecular layer of the Alzheimer's disease brain, a region devoid of neuronal perikarya [32]. Furthermore, neurons with excessive Abeta42 accumulation and cells that have apparently undergone a recent lysis are frequently observed in brain regions containing abundant amyloid plaques, are sparse in regions of low amyloid plaque density and have never been observed in comparable regions of age-matched control brains. There is also a clear inverse relationship between the local amyloid plaque density and local neuron density in any given Alzheimer's disease brain region. Further, there is a close relationship between the size of amyloid plaques and the size of surrounding neurons [153].

In addition, most amyloid plaques exhibit a remarkably consistent, spherical shape in the entorhinal cortex and hippocampus as revealed by serial sections of Abeta42 immunohistochemistry. This consistent spherical shape was not observed in the diffuse, vascular-associated amyloid plaques formed by extracellular deposition of Abeta42 [32].

In vitro experiments further support the origin of dense-core amyloid plaques from the over-accumulation of Abeta in neurons. Human neuroblastoma SK-N-MC cells, transfected with A7R, were exposed to 100 nanomolar Abeta42 for 6 hours leading to cell death [139]. Cytospin preparation of detached transfected cells and debris floating in the media after 12 hours of exposure to Abeta42 provide evidence that many cells had undergone lysis is shown by the presence of isolated (cytoplasm-free) nuclear remnants, the presence of aborted mitotic figures and the release of Abeta42-positive material into the culture media.

In in vivo experiments, mice were injected with pertussis toxin and FITC-labeled Abeta42 and, after 48 hours, FITC-labeled Abeta42 was observed in the neurons of the mouse brains [16] thereby providing in vivo evidence that vascular-derived Abeta42 can enter the brain, and then enter into the neurons. Additional studies (describe below) show that over time, these neurons accumulate pathological levels of amyloid leading to neuronal degeneration, synaptic decline, neuronal death (dense-core plaque formation), gliosis, and learning impairment.

In summary, the evidence is supportive of the inventor's lysis hypothesis whereby dense-core amyloid plaques in the Alzheimer's disease brains arising from the lysis of neurons overburdened by excessive intracellular deposition of Abeta peptide rather than the spontaneous extracellular aggregation or seeding of exogenous Abeta as per the amyloid hypothesis. The local release of active lysosomal enzymes, which persist within these plaques [14], degrades most of the released neuronal components (e.g., MAP-2), leaving behind in place those that are resistant to proteolytic digestion (e.g., neurofilament proteins, tau, ubiquitin, amyloid, mRNA, DNA, lipofuscin) as neuronal debris [28]. These data are also indicative of the source of the intracellular amyloid in the vascular smooth muscle and endothelial cells, as well as in the neurons, from the vascular system whereby the dysfunctional BBB allows unregulated amounts of Abeta into the brain (e.g., vascular-amyloid plaques) and then into neurons that internalize lethal amounts causing them to die producing the prototypical dense-core amyloid plaque. Hence, not all amyloid plaques are derived from dead neurons [24,28], but it is the dense-core, amyloid (dead neuron) plaques that lead to memory loss, mild cognitive impairment, neuroinflammation, and ultimately Alzheimer's disease. The other amyloid plaques types such as the diffuse amyloid plaque that represent areas of amyloid leakage near vessels, and those from Purkinje cells [174], do not appear to have a pathological consequence since they are not composed of neuronal material and are not associated with inflammatory cells. This invention provides methods to prevent leakage of vascular components such as Abeta into the brain, and to block entry of Abeta into cells such as the neurons before they accumulate lethal amounts.

Clinical trials using compounds to remove extracellular Abeta42 failed because they are trying to validate an inaccurate hypothesis, meaning that neurons do not produce enough amyloid to create the plaques to become toxic to other neurons, and some of the clinical data clearly showed that removing extracellular amyloid (that was efficacious in autopsied brains) had no bearing on cognition improvement. It is the amyloid that accumulates in the neurons that leads to their death. Thus, these clinical trials failed first because the neurons that cause the onset of Alzheimer's disease (and other dementias, and MCI) are already dead causing the symptoms, and second, because extracellular Abeta (e.g., diffuse plaques) is benign, removing extracellular amyloid will not prevent disease progression. In fact, despite achieving efficacy in removing extracellular amyloid in the autopsied brains of treated subjects in clinical trials using anti-amyloid antibody therapies, the clinical endpoints for cognitive improvement were not met because the neurons were already dead. Furthermore, the neurons will continue to die due to the pouring of vascular-derived Abeta into the brain to cause further neuronal degeneration, which will then continue to trigger neuroinflammation leading to additional neuronal death.

Although these clinical trials treating Abeta failed to meet their efficacy endpoints, using compounds to prevent BBB leakage, to reduce or prevent intraneuronal accumulation of vascular-derived Abeta, and to reduce or prevent neuroinflammation together in accordance with this invention provides a prophylactic therapeutic approach to prevent or delay onset of Alzheimer's disease as well as other dementias and MCI. Once the diagnosis of MCI is made (and possibly sooner if predictive testing becomes available), there is a therapeutic window of opportunity to intervene to 1) stop further pouring of amyloid from the vascular system into the brain, 2) prevent further intraneuronal accumulation of amyloid in neurons before lysis, and 3) suppress the ongoing processes of gliosis.

In the present invention, specific A7R binding agents are administered, not to augment A7R function, but rather to reduce and/or block the excessive toxic accumulations of vascular-derived amyloid from entering the neurons before they die. This unique therapeutic approach is to use specific A7R binding agents (novel or re-purpose the use of such failed A7R-specific binding agents used in various clinical trials such as but not limited to agonist, antagonist, inhibitors, positive allosteric modulators, etc.) to help prevent the progression of neuronal death. The A7Rs are highly expressed in the basal forebrain cholinergic neurons that project to the hippocampus and cortex of normal and Alzheimer's disease brains, brain areas that are innervated by the basal forebrain cholinergic neurons associated with memory and cognition and which exhibit Alzheimer's disease-related pathology [11,13,19,61,111,144,145,178], and correlate well with brain areas that exhibit neuritic, dense-core amyloid plaques in Alzheimer's disease. The A7Rs modulate calcium homeostasis and release of the neurotransmitter acetylcholine, which are 2 important parameters involved in cognition and memory. The inventor herein now believes that the A7R, a neuronal homopentameric cation channel that is highly permeable to Ca² [158], plays a role in the pathological accumulation of Abeta42 in cells that abundantly express this receptor [172, 173, 174]. The nAChRs are a family of ligand-gated ion channels that are widely distributed in the brain [13, 61, 142, 144]. A decreased number of nicotinic acetylcholine receptors, including the A7R, have been reported in specific regions of the Alzheimer's disease brain. This deficit occurs early in the course of the disease and correlates well with cognitive dysfunctions [6, 11, 56, 117, 142, 158, 180]. A7R also binds with high affinity to alpha-bungarotoxin, an A7R antagonist [15, 20, 130, 147, 148, 159]. Receptor binding studies have revealed that Abeta42 binds to the A7R with exceptionally high affinity (Ki values of 4.1 and 5.0 picomolar for rat and guinea pig receptors, and IC₅₀˜0.01 picomolar in A7R transfected human neuroblastoma [SK-N-MC] cells) when compared to that of Abeta40, and that this interaction can be inhibited by A7R ligands [172, 173]. The fact that the Abeta42/A7R complex resists detergent treatment and remains detectable in the complex formed by western analysis lends further support to the high-affinity nature of this interaction and suggests that the Abeta42/A7R complexes form on the surfaces of A7R-expressing cells (e.g., neurons, smooth muscle cells) and remains intact during Abeta42 internalization and accumulation [17,172].

Since Abeta42, a major component of amyloid plaques, binds with exceptionally high affinity to A7R and accumulates within the neurons of Alzheimer's disease brains, a validation study was performed to assess the role of this binding in facilitating intraneuronal accumulation of Abeta42. Consecutive section immunohistochemistry and digital imaging revealed the spatial relationship between Abeta42 and A7R in affected neurons of Alzheimer's disease brains. Results show that neurons containing substantial intracellular accumulations of Abeta42 invariably express relatively high levels of the A7R. Furthermore, this receptor is highly co-localized with Abeta42 within neurons of Alzheimer's disease brains using double immunohistochemical and immunofluorescence methods [30,139,172].

To experimentally test the possibility that the binding interaction between exogenous Abeta42 and the A7R facilitates internalization and intracellular accumulation of Abeta42 in Alzheimer's disease brains, the fates of exogenous Abeta42 and its interaction with the A7R in vitro were assessed using cultured, A7R-transfected SK-N-MC human neuroblastoma cells that express elevated levels of this receptor [139]. Abeta42 is internalized via endocytosis in A7R-transfected SK-N-MC cells and co-localizes with the A7R within intracellular deposits [139]. Transfected cells treated with 100 nanomolar of Abeta42 showed some accumulation of Abeta42-positive material within 30 minutes. Cells treated for 3 hours with 100 nanomolar Abeta42 possessed prominent, irregularly shaped Abeta42-positive deposits. Double-label immunofluorescence revealed that essentially all intracellular Abeta42-positive deposits in these cells also exhibited intense A7R immunoreactivity. Intracellular deposits containing both Abeta42 and A7R were observed as well. Cells treated with 100 nanomolar Abeta40 for 3 hours showed little detectable accumulation of this peptide. Treatment of cells with alpha-bungarotoxin (10 micromolar) for 1 hour inhibited Abeta42 accumulation in transfected cells. Abeta42 internalization and accumulation in transfected cells was also blocked by 2 micromolar phenylarsine oxide, an inhibitor of endocytosis, whereas the dimethyl sulfoxide vehicle (0.1%) had no effect.

Several A7R-specific compounds were screened for their ability to block or reduce the toxic accumulation of Abeta42 in cultured neurons through the A7R. SK-N-MC neurons (ATCC, HTB-1), a human neuroblastoma cell line, were cultured in 4-well chamber slides. Cells were grown in chamber slides in Medium 199 supplemented with 10% fetal bovine serum. Prior to treatment with exogenous Abeta42 peptides, cells were grown for 16 hours in Medium 199 containing reduced (0.1%) fetal bovine serum and then exposed to 100 nM of Abeta42 added to the same medium for up to 24 hours. Working solutions of Abeta42 were maintained at pH 7.5 to prevent spontaneous aggregation. Cells were grown to ˜50% to 60% confluency in each of the 4-welled culture slides. The A7R compounds (Table 1) were added simultaneously with the Abeta42 peptides and exposed for the following time points: 30 minutes, 1 hour, 2 hours, 4 hours and 6 hours. Thereafter, the cells were fixed with 4% paraformaldehyde in 0.1 sodium PBS for 30 minutes, then replaced with saline to be stored at 4° C. for immunocytochemistry (ICC) the next day.

The ICC methods have been previously described [139].

TABLE 1
Listing of the Experimental Compounds.

| Compound | Properties | Titer | | Vendor | Cat# | References |
|---|---|---|---|---|---|---|
| Alpha-bungarotoxin | high-affinity nicotinic | 1 | nM | Sigma | T0195 | Nagele et al, |
| | acetylcholine receptor | | | | | 2002^{20,139} |
| | antagonist | | | | | |
| Nicotine | Nicotinic acetylcholine | 10 | uM | Sigma | SML1236 | Olincy and |
| | receptor agonist | | | | | Stevens, 2007¹⁴³ |
| Varenicline | Full A7R agonist | 200 | uM | Sigma | PZ0004 | Mihalak et al, |
| | | | | | | 2006¹³² |
| GTS-21 | Selective A7R agonist | 10 | uM | Millipore | 505228 | Arendash et al, |
| | | | | | | 1995 |
| Methyllycaconitine | Potent and specific | 10 | nM | Sigma | M168 | Liu et al, |
| | nicotinic receptor | | | | | 2015¹²³ |
| | antagonist that | | | | | |
| | binds to neuronal | | | | | |
| | α-bungarotoxin | | | | | |
| | sites | | | | | |

After the treatment of the cells with the Abeta42 and compounds, the morphology of the cells were assessed using the semi-quantitative scheme (minimum of 100 cells counted) presented in Table 2.

TABLE 2
Morphological Scoring Key.

| Score | Morphology/distribution |
|---|---|
| 3 | Flat, larger nucleus, prominent processes: >90% of cells |
| 2 | Some signs of degeneration: <20% of cells |
| 1 | Balled-up cells, not very flat, lost adhesion, loss of |
| | prominent processes: >50% of cells |
| 0 | Mostly balled-up cells, atrophic, shriveled, loss of |
| | adhesion: >90% of cells |

Also, the cells were assayed to detect Abeta42 and then were analyzed for Abeta42 immunolabeling intensity and distribution using the semi-quantitative scale (minimum of 100 cells counted) presented in Table 3.

TABLE 3
Abeta42 Immunolabeling Key.

| Score | Immunolabeling intensity/distribution |
|---|---|
| 3 | Strong, prominent intracellular Abeta42 labeling: >75% of cells |
| 2 | Moderate, obvious intracellular Abeta42 labeling: ~50% of cells |
| 1 | Weak intracellular Abeta42 labeling: >75% of cells |
| 0 | No detectable intracellular Abeta42 labeling: >90% of cells |

The data from the experiments are presented in Table 4.

TABLE 4
Summary of the Morphological and Abeta42 Immunolabeling Scores.

| | Morphologic scores | | | | | Abeta42 immunolabeling scores | | | | |
|---|---|---|---|---|---|---|---|---|---|---|
| Compound + Abeta42 (100 nM) | 30 m^{a} | 1 hr^{a} | 2 hr^{b} | 4 hr^{b} | 6 hr^{c} | 30 m^{a} | 1 hr^{a} | 2 hr^{b} | 4 hr^{b} | 6 hr^{c} |
| Vehicle (water) | 3 | 2 | 1.8 | 2 | 1 | 2 | 1 | .3 | 1.2 | 1 |
| Alpha-bungarotoxin (1 nM) | 3 | 3 | 3 | 3 | 2.5 | 1 | 0/1 | 1/0 | 1/0 | 0 |
| Nicotine (10 nM) | 3 | 2 | 2.7 | 2.8 | 2.8 | 1 | 1 | 1.7 | 1 | 0.3 |
| Varenicline (200 uM) | 3 | 3 | 3 | 3 | 3 | 0 | 1 | 0 | 0.5 | 0 |
| GTS-21 (10 mM) | 3 | 3 | 2.7 | 2.5^{c} | 2.5 | 1 | 2 | 1 | 1.3^{c} | 0.5 |
| Methyllycaconitine (10 nM) | 2 | 2 | 2.7 | 2.5 | 2.8 | 2 | 1 | 0.8 | 0 | 0 |

Key:
^{a}= represents data from 1 experiment;
^{b}= represents average data from triplicate experiments;
^{c}= represents average data of duplicate experiments

Experiments were performed to determine the presence of A7R in the SK-N-MC human neuroblastoma cells. No detectable

Abeta42 labeling was detected in the untreated cells that appeared healthy as evident by the presence of euchromatic nuclei, mitotic cells, and prominent nucleoli. However, when treated with Abeta42, intracellular Abeta42 was detected in cells that were morphologically degenerating and atrophic, among the presence of other Abeta42-positive apoptotic/dying cells. Cells treated with Varenicline (A7R agonist) and Abeta42 were protected from Abeta42 toxicity, as the observed cells were healthy, with several mitotic cells. Similar results were obtained when cells were treated with nicotine (A7R agonist) and Abeta42, methyllycaconitine (A7R antagonist) and Abeta42, and GTS-21 and Abeta42. Generally, similar results were also obtained at other time points (30 minutes, 1 hour, 2 hours, and 6 hours). Experiments for 30 minutes and 1 hour were only performed 1 time, experiments for 2 hours and 4 hours were performed in triplicates, experiments for 6 hours were performed in duplicates.

In summary, the histopathological and in vitro experimental data provide evidence that Abeta gains entry via endocytosis through the A7R into A7R-positive cells. Over time, as the cells accumulate toxic levels of Abeta42, they degenerate leading to their cellular debris. Importantly, the neurons can be protected from accumulating toxic amounts of Abeta42 by blocking its entry through the A7R by using several classes of A7R compounds (e.g., agonist, antagonist, etc.).

The present invention thus provides methods and dosing regimens which block or reduce the toxic accumulation of amyloid in A7R-positive cells such as, but not limited to neurons, smooth muscle cells, and endothelial cells, through the use of specific A7R binding agents.

The inventors herein now believe that the high-affinity binding of Abeta42 to A7R on neuronal surfaces that express this receptor is an important early step that facilitates internalization and gradual accumulation of Abeta42 in neurons of Alzheimer's disease brains. Unlike prior teachings relating to A7R agents [19,30,39,86,87,88,89,90,91,92,93,94,95,96,105, 111], in the present invention the A7R agents are not used to activate or inhibit function of the receptor. Instead, in the present invention, A7R agents including, but not limited to, agonists, antagonists, inhibitors, and positive allosteric modulators, are used to block the toxic accumulation of the vascular-derived amyloid Abeta that pour into the brain from a dysfunction of the BBB, from binding and entering cells, especially the neurons of the brain. Thus saving neurons and other cells such as smooth muscle cells and endothelial cells before they die is a novel application for A7R specific binding agents.

Neuroinflammation or gliosis has been described in the brains of people with Alzheimer's disease. Gliosis is predominantly produced by the activity of microglia and astrocytes in the brain. The microglia inflammatory cells of the brain constantly search the brain for cell debris and infectious agents, while the primary function of other supportive cells, the astrocytes, are to maintain the BBB while repairing injured areas by extending their processes that eventually form a glial scar. The initial response includes the migration of the microglia to the site of the injury, followed by the production of a dense fibrous network of astrocytic processes producing the glial scar to isolate and sequester the damage from the unaffected areas in the brain.

As noted in the background section, neuroinflammation was believed in the 1980s to be the cause of neuronal death in the Alzheimer's disease brain. Further it was believed that all extracellular amyloid triggers gliosis. In an effort to study gliosis in the Alzheimer's disease brain, a triple-immunohistochemical method was designed by the inventor to simultaneously observe the presence of inflammatory cells (e.g., astrocytes, microglia) among the various types of amyloid plaques [34]. An association of purple-stained reactive microglia and black-labeled reactive astrocytes with red-labeled Abeta42-positive dense-core plaque was observed in serially-sectioned, Alzheimer's disease cortical tissues. Reactive microglia were observed toward the core of these dense-core amyloid plaques. Although no gliosis was associated with the diffuse-type amyloid plaques, most of the dense-core, amyloid plaques were associated with inflammation but the extent of microglial and astrocytic activation varied, as some of them were only associated with activated microglia suggesting that the processes of neuroinflammation begin with the recruitment of activated microglia. In contrast to the belief that extracellular amyloid triggers gliosis, microglia were seldom detected on the plaque periphery and appeared to bypass the amyloid-ridden plaque corona to migrate deep within the dense cores of the amyloid plaques. This observation implies that something within the amyloid plaque core, perhaps within the dead neuron, such as nucleic acids rather than the dispersed amyloid attracts microglia. A most likely candidate could be the neuronal nuclear DNA fragments as the released adenosine tri-phosphate or adenosine di-phosphate could induce microglial chemotaxis via the Gi/o-coupled P2Y receptors toward the center of these plaque types. Therefore, the role of the microglia would be to ingest such critical nuclear debris. Microglia also have receptors such as P2X, and the scavenger receptor A (CD36) on their membrane that are activated by purines or fragmented DNA that cause the microglia to be chemoattractant. To provide further support, the presence of purple-labeled reactive microglia was closely associated with the red-labeled, NeuN-positive, neuronal nuclei in many plaque areas in cortical tissues of Alzheimer's disease using double immunohistochemical methods [34].

Astrocytes become activated secondarily perhaps via microglia-derived interleukin-1 [55]. Reactive astrocytes respond by extending their processes deep into the amyloid plaques. Abeta42-positive immunoreactivity was also detected in plaque-associated astrocytes indicating that these cells may be phagocytizing the dispersed plaque Abeta42 material. Interestingly, the results show that some astrocytes containing abundant Abeta42-positive deposits also undergo lysis, resulting in the formation of astrocyte-derived amyloid plaques (another plaque type) in the cortical molecular layer in brain regions showing moderate to advanced Alzheimer's disease pathology [138]. Other astrocytes in areas with little dense-core amyloid plaques do not possess the activated GFAP-positive morphology nor detectable intracellular Abeta, suggests that activation must be a local rather than a systemic event. Interestingly, the presence of astrocytes inhibits the ability microglia to ingest the Abeta in vitro [40]. Taken together, the function of subsequent astrocyte activation may be to modulate or regulate the microglia activity.

In summary, disclosed herein is a mechanism where the initial death of neurons in the Alzheimer's brain begins from the over-accumulation of intracellular, vascular-derived Abeta. Once the cell dies, it incidentally releases its contents, some of which activate the microglia to mobilize to the area to ingest or phagocytize the cellular debris. While the microglia are present, they then release factors that activate the local astrocytes to extend their processes in order to create a scar that appears like a web as it tries to fill in the hole (as evident by the missing MAP-2 immunolabeling as described above) left from the dead neuron. Interestingly, those same local astrocytes then release factors to deactivate the microglia. Unfortunately, those released factors, which may be specific to the astrocytes or microglia, also harm neighboring neurons causing them to die as collateral damage from the processes of inflammation; thereby, creating an uncontrolled cascade of pathological events [24].

The present invention also provides methods and dosing regimens to reduce or minimize neuroinflammation triggered by neuronal death that becomes the dense-core amyloid plaque. The limited efficacy of NS AIDs to treat subjects with Alzheimer's disease may also lie in the inability to suppress the critical pathological events, which are the breaching of the BBB, and the lysing of the Abeta-overburdened neurons. However, the benefits of anti-inflammatory agents such as, but not limited to, steroids and/or NS AID therapies would help offset subsequent secondary neuronal death due to the secreted factors from the activated microglia and reactive astrocytes. Even as far back as 1898, it was believed that plaques corresponded to a modified type of glial cell, mostly due to the presence of fibrous material. It was then concluded that glial cell proliferation was a secondary, not primary event to nerve cell degeneration.

Furthermore, the onset of Alzheimer's disease coincides with the detection of inflammatory markers around amyloid plaques and dystrophic neurites. As noted, these CNS-inflammatory cells (microglia and astrocytes) secrete a number of factors that can unfortunately harm local functioning neurons. This notion is based on sets of reports that support the idea that altered patterns in the glia-neuron interactions constitute early molecular events leading to neurodegeneration in Alzheimer's disease [154]. A direct correlation has been established between the Abeta-induced neurodegeneration and cytokine production, and its subsequent release. Neuroinflammation is responsible for an abnormal secretion of proinflammatory cytokines, chemokines, and complement activation products from the resident CNS cells that trigger signaling pathways and play a relevant role in the pathogenesis of the inflammatory process occurring during the development of the pathology because of their chemotactic activity on brain phagocytes [122,126,154,186].

Once the neurodegeneration cascade is initiated, microglial and astrocytes may play major roles directly and indirectly promoting self-sustaining neurodegeneration cycles [27,121,122,160].

Clinical trials with anti-inflammatory agents produced unsatisfactory results. As described herein, these processes are secondary to the dying neurons, and so, these clinical trials may have had unrealistic expectations of cognitive improvement when other pathologies are not treated. Such interventions may have helped to slow down the progression due to the potentially destructive secondary consequences of the inflammatory cells leading to subsequent pathologies, but since Alzheimer's disease is irreversible, the benefit was not observable. In a recent study that followed 247 Alzheimer's disease subjects over 13 years, neuroinflammation was independently linked to early death, thereby rapidly advancing the disease [140]. This study suggested that inflammation, not amyloid or tau pathology, was an independent underlying mechanism in Alzheimer's disease neuropathology, supporting efficacy of the methods and dosing regimens in this invention. These findings indicate that the anti-inflammatory agents can be helpful in the prevention, and perhaps averting further cognitive decline, but not in the treatment of Alzheimer's disease as it is too late in the pathological process identified in this invention since neuronal death leads to Alzheimer's disease and subsequent gliosis [24,27,46].

Those clinical trials using anti-inflammatory compounds have failed because neuroinflammation is a consequence of neuronal death, and neuronal death is a consequence of a dysfunctional BBB. Although these clinical trials failed, such compounds to prevent or reduce neuroinflammation will prevent or delay onset of Alzheimer's disease and other dementias and MCI when used in accordance with the present invention in a prophylactic therapeutic approach and in combination with agents which block over-accumulation of Abeta into neurons via A7R and agents which preventing unregulated entry of vascular-derived amyloid through a dysfunctional BBB into the brain. Once the diagnosis of MCI is made (or sooner when methods to identify subjects at risk to develop MCI are developed), there is a therapeutic window of opportunity to intervene to 1) stop further pouring of amyloid from the vascular system into the brain, 2) prevent further intraneuronal accumulation of amyloid before they lysis, and 3) suppress the ongoing processes of gliosis.

The inventors herein believe that Alzheimer's disease begins with cardiovascular pathology that leads to entry of unregulated amyloid into the brain. The amyloid then binds to A7R neuronal receptors and leads to toxic levels of intraneuronal amyloid causing cell death. Accordingly, most successful intervention with the present invention will occur before a subject is diagnosed with Alzheimer's disease.

In one nonlimiting embodiment, the agent targeting A7Rs along with agents to prevent and/or control BBB leakage and to minimize and/or inhibit neuroinflammation are administered to a subject prior to the onset of Alzheimer's disease. For example, the term MCI is used to describe a state of cognitive decline representing a transition between normal cognition and dementia [68, 113]. This state is characterized by impairment in memory and other cognitive functions as demonstrated by standardized neuropsychological tests. A substantial percentage of subjects with the amnestic form of MCI progress to Alzheimer's disease within 4 years of diagnosis and 50% of those diagnosed with MCI go on to develop dementia, according to NICE (National Institute for Health and Care Excellence) guidelines. In one nonlimiting embodiment of the present invention, the agent targeting A7Rs is administered with an agent to prevent and/or control BBB leakage and an agent to minimize and/or inhibit neuroinflammation to a subject diagnosed with MCI. However, if test(s) become available to predict subjects at risk for MCI, then the 3-prong, prophylactic therapeutic approach defined in this invention would be administered to prevent the onset of MCI. In another nonlimiting embodiment, the agent targeting A7Rs is administered with an agent to prevent or control BBB leakage, and an agent to minimize or inhibit neuroinflammation to a subject diagnosed with dementia.

A memory assessment service is useful as a single point of referral for all subjects with a suspected diagnosis of dementia.

In one nonlimiting embodiment, an agent targeting A7Rs, an agent to prevent and/or control BBB leakage, and an agent to minimize and/or inhibit neuroinflammation are administered to specific subject populations at risk for development of Alzheimer's disease. For example, individuals with Down syndrome have an increased risk of Alzheimer's disease. Estimates suggest that 50 percent or more of people with Down syndrome will develop dementia due to Alzheimer's disease as they age and virtually all individuals with Down syndrome develop sufficient neuropathology for a diagnosis of Alzheimer's disease by the age of 40 years. The Abeta peptide has been found in the brains of children with Down syndrome as young as 8 years, and the deposits increase with age. People with Down syndrome begin to show symptoms of Alzheimer's disease in their 50s or 60s [69,75,79,80,81,85]. There are varying accounts of the age of onset, but generally between the ages of 45 to 50 years old, between 30% to 40% are diagnosed with Alzheimer's disease. By the time they are in their 60s, the number is closer to 50% to 77%. Alzheimer's disease is responsible for the sharp decline in survival in persons with Down syndrome older than 45 years. The time from the first symptoms of Alzheimer's disease to death is usually about 9 years [73, Error! Reference source not found.] . Hence, Down syndrome individuals offer a shorter duration to test this invention.

Accordingly, an aspect of the present invention relates to administration of an A7R agent with agents to prevent and/or control BBB leakage and to minimize and/or inhibit neuroinflammation to a subject with Down syndrome to prevent, inhibit or delay onset of Alzheimer's disease in the subject. In one nonlimiting embodiment, administration of A7R agents with agents to prevent and/or control BBB leakage and to minimize and/or inhibit neuroinflammation to treat Alzheimer's disease in Down syndrome individuals will occur after diagnosed with MCI.

For purposes of the present invention, A7R agents may be used alone to prevent or decrease levels of intracellular amyloid in the neurons of the brain to save them from degenerating and dying, or in combination with other medications such as, but not limited to, agents for cardiovascular pathology which minimize BBB leakage, and/or agents to reduce neuroinflammation in the brain activated from neuronal death. Nonlimiting examples of agents, which can be used in combination with an A7R agent in accordance with the present invention include, but are not limited to medications for treatment of atherosclerosis, high blood pressure, hypertension, and stroke such as angiotensin-converting enzyme inhibitors, aldosterone inhibitors, angiotensin II receptor blockers, beta-blockers, cholesterol-lowering drugs, and low dose Natrexon. Combination therapies may be administered at the same time or at different times to the subject.

Pharmaceutical compositions or formulations for use in the present invention include those suitable for oral, rectal, nasal, topical (including buccal and sub-lingual), vaginal or parenteral (including intramuscular, subcutaneous and intravenous) administration or in a form suitable for administration by inhalation or insufflation.

An A7R binding agent, together with a conventional adjuvant, carrier, or diluent, alone or in combination with other medications as described herein, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use.

Such pharmaceutical compositions and unit dosage forms of may further comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to one hundred (100) milligrams, per tablet, are accordingly suitable representative unit dosage forms. In one nonlimiting embodiment, a dosage of 10 to 25 milligrams is administered once per day.

The compounds of the present invention can be administered in a wide variety of oral and parenteral dosage forms.

For example, for preparing pharmaceutical compositions from the compounds of the present invention, pharmaceutically acceptable carriers can be either solid or liquid. Solid form preparations include powders, tablets, pills, capsules, cachets, suppositories, and dispensable granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided A7R binding agent alone or in combination with other medications as described herein.

In tablets, the A7R binding agent alone or in combination with other medications as described herein is mixed with the carrier having the necessary binding capacity in suitable proportions and compacted in the shape and size desired. The powders and tablets preferably contain from 5 or 10 to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the A7R binding agent alone or in combination with other medications as described herein is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, such as water or water-propylene glycol solutions. In addition, parenteral injection liquid preparations can be formulated as solutions in aqueous polyethylene glycol solution.

Sterile liquid form compositions include sterile solutions, suspensions, emulsions, syrups and elixirs. The A7R binding agent can be dissolved or suspended in a pharmaceutically acceptable carrier, such as sterile water, sterile organic solvent or a mixture of both.

The A7R binding agents alone or in combination with other medications as described herein can thus be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the A7R binding agent alone or in combination with other medications as described herein can be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the A7R binding agent alone or in combination with other medications as described herein in water and adding suitable colorants, flavors, stabilizing and thickening agents, as desired.

Aqueous suspensions suitable for oral use can be made by dispersing the finely divided A7R binding agent alone or in combination with other medications as described herein in water with viscous material, such as natural or synthetic gums, resins, methylcellulose, sodium carboxymethylcellulose, or other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, and solubilizing agents.

For topical administration to the epidermis the A7R binding agent alone or in combination with other medications as described herein may be formulated as an ointment, cream or lotion, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising an A7R binding agent alone or in combination with other medications as described herein in a flavored base, usually sucrose and acacia or tragacanth; pastilles comprising the A7R binding agent alone or in combination with other medications as described herein in an inert base such as gelatin and glycerin or sucrose and acacia; and mouthwashes comprising the A7R binding agent alone or in combination with other medications as described herein in a suitable liquid carrier.

Solutions or suspensions can also be applied directly to the nasal cavity by conventional means, for example with a dropper, pipette or spray. The formulations may be provided in single or multi-dose form. In the latter case of a dropper or pipette, this may be achieved by the subject administering an appropriate, predetermined volume of the solution or suspension. In the case of a spray, this may be achieved for example by means of a metering atomizing spray pump. To improve nasal delivery and retention the A7R binding agent alone or in combination with other medications as described herein may be encapsulated with cyclodextrins, or formulated with other agents expected to enhance delivery and retention in the nasal mucosa.

Administration to the respiratory tract may also be achieved by means of an aerosol formulation in which the A7R binding agent alone or in combination with other medications as described herein is provided in a pressurized pack with a suitable propellant such as a chlorofluorocarbon, for example dichlorodifluoromethane, trichlorofluoromethane, or dichlorotetrafluoroethane, carbon dioxide, or other suitable gas. The aerosol may conveniently also contain a surfactant such as lecithin. The dose may be controlled by provision of a metered valve.

Alternatively the A7R binding agent alone or in combination with other medications as described herein may be provided in the form of a dry powder, for example a powder mix of the compound in a suitable powder base such as lactose, starch, starch derivatives such as hydroxypropylmethyl cellulose and polyvinylpyrrolidone. Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 5 to 10 microns or less. Such a particle size may be obtained by means known in the art, for example by micronization.

Formulations adapted to give sustained release of the A7R binding agent alone or in combination with other medications as described herein may also be employed.

Pharmaceutical preparations are preferably in unit dosage forms. In such form, the preparation is subdivided into unit doses containing appropriate quantities of the A7R binding agent alone or in combination with other medications as described herein. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The amount of the A7R binding agent to be administered may be in the range from about 1 mg to 2000 mg per day, depending on the activity of the A7R binding agent and the subject being treated.

Clinical trials targeting the A7R have failed because of an inaccurate efficacy endpoint of activating the receptor to improve cognition. However, using such A7R-specific compounds to prevent the over-accumulation of Abeta to save the neurons from death will prevent and/or delay onset of Alzheimer's disease as well as other dementias and MCI using this prophylactic therapeutic approach, but only in combination with the other 2 therapeutic targets noted in this invention (e.g., vascular leakiness, and neuroinflammation). Once the diagnosis of MCI is made, there is a therapeutic window of opportunity to intervene to 1) stop further pouring of amyloid from the vascular system into the brain, 2) prevent further intraneuronal accumulation of amyloid before they lysis, and 3) suppress the ongoing processes of gliosis.

Another aspect of this invention relates to assessing BBB health through examinations of the BRB to enrich targeted populations based on Alzheimer's disease vascular risk factors. Data suggest that the BRB is dysfunctional in eye pathologies [114,171], and that there is an association with vascular diseases [184], which is again a risk factor for Alzheimer's disease [167]. Endothelial damage may actually be the primary event on BBB and BRB dysfunction suggesting that the primary pathological event may occur from outside the brain for such diseases of the CNS. Endothelial damage is also a primary event in diabetic retinopathy as BRB breakdown precedes pathological retinopathy in diabetes [22,170,171]. Vascular pathologies “precede” the presence of plaques and cognitive impairments in animal transgenic Alzheimer's disease mouse models [165]. In addition to the detection of amyloid in the cerebrovasculature, which is particularly present in the leptomeningeal and cortical arteries resulting in cerebral amyloid angiopathy, it was also determined that amyloid is targeted to the vasculature in a mouse model of hereditary cerebral hemorrhage with amyloidosis [62,171]. There is also a positive correlation between retinal pathology and Alzheimer's disease [10]. Alzheimer's disease subjects often exhibit poor vision and others show visual signs of impairment [9,18,21,110]. In one nonlimiting embodiment, high resolution scans of the retina are to be used to assess the health of the BRB as a predictive biomarker of the health of the BBB, as the brain and the eye have similar anatomical vascular barrier structures. Beyond the current standard fundus photography, non-invasive methods of optical coherence tomography providing microaneurysm counts, assessment of length and diameter of retinal vessels, and computerized quantification of all pathological elements may also be useful as diagnostic tools and/or efficacy end points [125]. The first 40 subjects in a 200-participant study showed that retinal changes strongly correlated with amyloid plaque development in the brain [175]. Furthermore, assessing the permeability of the BRB and BBB via detection of sucrose and albumin can also be used in combination with imaging data [47].

In another study, the level of Abeta in the eye significantly correlated with the burden of Abeta in the brain and allowed researchers to accurately identify people with Alzheimer's disease [97]. Accordingly, detection of Abeta or other vascular elements (e.g., immunoglobulins) in the eye, as an indicator of BRB and BBB health, may be used in the present invention to identify those at risk to develop MCI, Alzheimer's disease, and other dementias. Those at risk would be candidates for administration of an A7R binding agent with agents to prevent and/or control BBB leakage and to minimize and/or inhibit neuroinflammation.

Other subject populations at risk for Alzheimer's disease to which the A7R binding agent with agents to prevent or control BBB leakage and to minimize or inhibit neuroinflammation may be administered in accordance with the present invention include, but are not limited to, subjects with diabetes, high blood pressure, and vascular diseases as well as individuals with a genetic predisposition which may be indicated by biomarkers such as, but not limited to ApoE (APO4 gene), ABCA7, CLU, CR1, PICALM, PLD3, TREM2 and/or SORL1.

In one nonlimiting embodiment, the agent targeting A7Rs with agents to prevent and/or control BBB leakage and to minimize and/or inhibit neuroinflammation are administered to a subject prior to the onset of Alzheimer's disease identified to be at risk via assessment of the health of the BBB by imaging and/or by assays. A leaky BBB is indicative of subjects who are at risk for Alzheimer's disease.

In addition to using retinal imaging as invaluable biomarker to indirectly assess the integrity of the BBB, other cardiovascular indicators such as high blood pressure, history of stroke, presence of atherosclerosis, etc. imply the importance of cardiovascular health as important risk factors for AD.

Assessing neuronal death is somewhat determined through clinical cognitive testing and other behavior examinations, but could also be validated by detecting neuronal debris in the fluids of the body (e.g., blood, cerebral spinal fluid), and if sensitive enough, at the earlier process of neuronal death well before clinical presentation is exhibited. For example, the expression of MAP-2 is missing in areas of the dense-core, senile amyloid plaques due to neuronal lysis [29]. The loss of MAP-2 labeling could be explained in 2 ways: either the antigen of the MAP-2 is modified by neuronal lysis to become unrecognizable by the primary antibody leading to the lack of IHC labeling, or the MAP-2 was digested and is missing in the area of the neuronal debris. If the latter, then it is equally possible that fragments of MAP-2 could be detected in the cerebral spinal fluid and/or vascular system as a biomarker or indication of neuronal death. The detection of (auto)-antibodies to fragments of MAP-2 and to other neuronal debris should provide a means to assess neuronal death as a diagnostic and potentially prognostic biomarker.

In summary, this invention describes how to identify individuals prone to an Alzheimer's disease diagnosis that begins with a diagnosis of MCI or a diagnosis of risk for MCI once available. Inclusion criteria would include individuals with MCI or those at risk for MCI, the APOE4 gene, BRB leakage, serum markers of neuronal debris, and vascular pathological risk factors. Down syndrome individuals offer a shorter duration to test this invention.

Alzheimer's disease is not reversible and therefore, a prophylactic therapeutic approach is required to prevent the onset of Alzheimer's disease (and other dementias, and MCI) as early as possible, perhaps at the onset of MCI or even before the diagnosis of MCI when tests become available to define individuals at risk.

As presented in this invention, the presence of Abeta high-affinity receptors on neurons suggests that CNS levels of Abeta are highly regulated. A series of Abeta42-stained sections of Alzheimer's brain tissues led to the hypothesis that neurons degenerate due to the over-accumulation of Abeta42 that has subsequently been validated through in vitro and in vivo experiments. If vascular-derived Abeta continues to pour into the brain due to a dysfunction vascular system, then over time, the neurons essentially over-engorge themselves to death, thereby lysing to form the dense-core amyloid plaque triggering neuroinflammation. This novel mechanism can explain why vascular pathology is an early event, why cognitive impairment occurs subsequently, why all clinical trials to date have failed, and why Alzheimer's disease is irreversible.

Subjects most likely to benefit from the invention will be identified through those biomarkers and imaging studies described in this invention. The pharmaceutical preparations of the compounds according to the present invention will be co-administered with one or more other active agents in combination therapy to prevent BBB leakage of amyloid into the brain, to prevent the over-accumulation of vascular-derived amyloid into the neurons by blocking enter through A7R compounds, and to prevent or minimal neuroinflammation. For example, the pharmaceutical preparation of the active compound may be co-administered (for example, separately, concurrently or sequentially), with one or more medications for treatment of atherosclerosis, high blood pressure, hypertension, or stroke such as angiotensin-converting enzyme inhibitors, aldosterone inhibitors, angiotensin II receptor blockers, beta-blockers, and cholesterol-lowering drugs, along with anti-inflammatory agents.

The following nonlimiting examples are provided to further illustrate the present invention.

### EXAMPLES

### Example 1: Learning Impairment Produced in Mice Treated with Abeta42 and Pertussis Toxin

This 2-week study was comprised of 2 groups of C57BL/6J mice as outlined in Table 5. Mice were infused with 100 μl pertussis toxin (3.0×10-3 μg/μl in saline) or 100 μl saline into the tail vein according to Table 5 to affect the BBB. Subsequently, Abeta42 (100 μl of 6.9 μM in saline) was infused into the tail vein. Groups 1 and 2 received 2 cycles of Abeta42 treatment.

TABLE 5
2-week Study Plan

| | | | | | | Behavioral | |
|---|---|---|---|---|---|---|---|
| | | | Age | | Abeta42 | assessment | Sacrifice |
| Gr | N | Genotype | (m) | PT/saline days | (days) | (days) | (day) |
| T | 4 | C57BL/6J | 12 | PT: 1, 3, 8, 10 | 5, 12 | CognitionWall | 15 |
| | | | | | | (14-15) | |
| C | 4 | C57BL/6J | 12 | Saline: 1, 3, 8, 10 | 5, 12 | CognitionWall | 15 |
| | | | | | | (14-15) | |

Key: PT = pertussis toxin; T(treated) = mice treated Abeta42 and pertussis toxin; C(control) = mice treated with Abeta42; N = sample size.

Deficits in learning behavior correlated with degenerating neurons in mice treated with pertussis toxin and Abeta42 as compared with untreated mice in the 2-week study. The CognitionWall discrimination learning test was used to test learning behaviors in the mice. Fifteen minutes before the start of the discrimination learning (D L) and reversal learning (RL) task at 16.30 h on the 3th light phase in the Phenotyper, the CognitionWall was placed in front of the reward dispenser spout. After placement, several free rewards were dispensed and standard chow was removed from the feeding station. Mice had to learn to earn their food (Dustless Precision Pellets, 14 mg) by going through the left hole in the wall for the next 2 days (D L1 and D L2). The middle and right hole were deemed incorrect holes and passing through these holes was without any consequences. During the subsequent 2 days, the rewarded hole was switched to the right hole (reversal learning; RL1 and RL2). During D L and RL, one reward was delivered for every fifth entry through the correct hole (FR5 schedule of reinforcement). Mice were not required to make 5 consecutive correct entries (i.e., no chaining requirement). The FR5 schedule was chosen after an initial pilot experiment showed that lower ratios resulted in satiety, as indicated by accumulation of non-consumed rewards in the cage. Online display of the number of earned rewards was used to evaluate food intake during the experiment. Based on pilot experiments to quantify the number of food rewards required to maintain body weight, mice were fed extra reward pellets when they earn fewer than 100 rewards per day for 2 or more consecutive days. The primary outcome measure included the number of entries required to reach the learning criterion of 80% correct entrances, computed as a moving average of the last 30 entries, and was taken as primary measure of learning rate both during initial discrimination learning as during the reversal learning stage of the task. Since a mouse may not learn this task, leading to censored data, a survival analyses is used to plot and statistically evaluate the data. Numerous additional informative measures were generated to better understand the behavior during the tasks, such as the total number of entries through any of the holes that may be taken as measure of activity, but those measures are not used to assess cognitive performance.

TABLE 6
Dosing notes and Cognitive Wall testing data (entries to 80%

| | Day 1 | Day 3 | Day 5 | Day 8 | Day 10 | Day 12 | |
|---|---|---|---|---|---|---|---|
| # | (PT) | (PT) | (Abeta42) | (PT) | (PT) | (Abeta42) | Entries |
| T1 | OK | OK | OK | OK | OK | SC | 129 |
| T2 | OK | OK | OK | OK* | OK* | SC | 310 |
| T3 | OK | OK | OK | OK | OK | OK | 182 |
| T4 | OK | OK | OK | OK* | OK | OK* | NA |
| T5 | OK | OK | +/− | OK* | OK | OK | NA |
| T6 | OK | OK | +/− | OK | OK | OK | 476 |
| C1 | OK | OK | OK | OK | OK | OK | 307 |
| C2 | OK | OK | +/− | OK | OK | OK | 130 |
| C3 | OK | OK | +/− | OK | OK | OK | 180 |
| C4 | OK | OK | OK | OK | OK* | OK | 264 |
| C5 | OK | OK | OK | OK | OK | OK | 134 |
| C6 | OK | OK | OK | OK | OK | OK | 370 |

Key: PT = pertussis toxin; T(treated) = mice treated Abeta42 and pertussis toxin; C(control) = mice treated with Abeta42; OK* = indicates that the needle was mis-localized in first instance, but OK after placing it a second time and infusing; +/− = indicates that a part of the solution was expected to be by injected s.c.; SC = indicates that the entire volume was most likely injected subcutaneous; NA = too impaired to test.

Mice had to learn to earn their food by going through the left hole in the wall. Although the typical sample sizes used in this test are 12-16 mice to power a study to reach conclusive results, this 2-week pilot test only used 6 mice per group to establish future study parameters and therefore statistical significance was not expected. Furthermore, 2 of the 6 PT-treated mice (T1, T2) did not receive their second Abeta42 dose Table 6). Nevertheless there was a trend towards a decreased performance of the PT/Abeta42-treated group in terms of an increased number of entries required to reach the learning criterion (G-rho weighted log-rank test p=0.09). Most importantly 2 of the 6 PT/Abeta42-treated mice were so impaired that they could not be tested. In an effort to quantitate the 2 NA scores, if they were estimated to score ˜500, slightly above the worse PT/Abeta42-treated score, then it would indicate that PT/Abeta42 treatment would increase entries by 50% (-230 entries of Abeta42-treated mice as compared with -350 entries of the PT/Abeta42-treated mice; Student's T-Test=p<0.008).

This data suggested that expanding the study with larger cohorts would indicate that PT treatment in combination with Abeta42 injections would lead to deficits in learning. The data also suggested that Abeta42 infusions without pertussis toxin did not impact learning impairment and that naive mice would produce less entries and therefore no learning impairment.

### Example 2: Learning Impairment Produced in Mice Treated with Abeta42 and Pertussis Toxin

Data from the 2-week pilot study (Example 1) indicated the need to extend the study to test longer time periods to expand the analyses to investigate other learning models (e.g., the nesting test and Morris water maze test), and to investigate immunohistochemical markers (e.g., synaptophysin, mouse immunoglobulin, Abeta42).

The study design is presented in Table 7.

TABLE 7
9-week Study Design

| | | FITC-Abeta42/ | | |
|---|---|---|---|---|
| | PT/saline | saline | Behavioral assessment | Sacrifice |
| Gr | (days) | (days) | (days) | (day) |
| 1 | Saline: 1, 3, | Saline 5, | Nesting Test (21, 42, 63) | 22 (n = 10) |
| | 8, 10, 22, 24, | 12, 26, 33, | CognitionWall (21-22, 42-43, 63-64) | 43 (n = 10) |
| | 29, 31, 43, | 47, 54 | Water maze (21, 42, 63) | 64 (n = 10) |
| | 45, 50, 52 | | | |
| 2 | Saline: 1, 3, | Abeta425, | Nesting Test (21, 42, 63) | 22 (n = 10) |
| | 8, 10, 22, 24, | 12, 26, 33, | CognitionWall (21-22, 42-43, 63-64) | 43 (n = 10) |
| | 29, 31, 43, | 47, 54 | Water maze (21, 42, 63) | 64 (n = 10) |
| | 45, 50, 52 | | | |
| 3 | PT: 1, 3, 8, | Saline 5, | Nesting Test (21, 42, 63) | 22 (n = 10) |
| | 10, 22, 24, | 12, 26, 33, | CognitionWall (21-22, 42-43, 63-64) | 43 (n = 10) |
| | 29, 31, 43, | 47, 54 | Water maze (21, 42, 63) | 64 (n = 10) |
| | 45, 50, 52 | | | |
| 4 | PT: 1, 3, 8, | Abeta425, | Nesting Test (21, 42, 63) | 22 (n = 10) |
| | 10, 22, 24, | 12, 26, 33, | CognitionWall (21-22, 42-43, 63-64) | 43 (n = 10) |
| | 29, 31, 43, | 47, 54 | Water maze (21, 42, 63) | 64 (n = 10) |
| | 45, 50, 52 | | | |

Key: PT = pertussis toxin; All groups with 20 C57BL/6J 1-year old mice.

Nine-week and 6 month learning studies are conducted to confirm that over time, vascular-derived Abeta will lead to learning impairment as further tested by the nesting test and the Morris water maze in addition to the established Cognitive Wall test (see Example 1). The mice are tested to assess nest-building behavior, a reported sensitive test of learning. In this test, additional nesting material (Nestlet of 3 gram compressed cotton) is introduced into each animal's home-cage approximately 3 hours before the start of the dark phase. The next morning, nest-building behavior is scored according to a previously described rating scale of 1-5 [179]:1=Nestlet >90% intact, 2=Nestlet 50%-90% intact, 3=Nestlet mostly shredded but no identifiable nest site, 4=identifiable but flat nest, 5=crater-shaped nest. Impaired nest-building behaviors are expected in mice treated with pertussis toxin and Abeta42 (p<0.01).

Spatial memory is tested in a Morris water maze setup. Before testing, mice are handled for at least 5 days, until they do not try to jump of or walk from the experimenter's hand. A circular pool (125 cm) which is painted white with non-toxic paint is filled with water (30 cm below the rim) and kept at a temperature of 25° C. An escape platform (ø9 cm) is placed at 30 cm from the edge of the pool submerged 1 cm below the water surface. Visual cues are located around the pool at a distance of ˜1.5 m. During testing lights are dimmed and covered with white sheets and mice are video-tracked using ViewerII (Viewer 2, BIOBSERVE GmbH, Bonn, Germany). Mice are trained for 5 consecutive days, 2 sessions of 2 trials per day with a 1 minute to 3 minute inter-session interval. In each trial, mice are first placed on the platform for 30s, and then placed in the water at a random start position and allowed a maximum of 60 seconds to find the platform. Mice that are unable to find the platform within 60 seconds are placed back on the platform by hand. Within each 2-trial session, after 30 seconds on the platform mice are tested again. On day 5 or day 6 a probe trial is performed with the platform removed. Mice are placed in the pool opposite from the platform location and allowed to swim for 60s. During training trials, the latency, distance and speed to reach the platform are measured; in the probe trial, the time spent and distance traveled in each quadrant of the pool are measured, as well as the number of platform-zone crossings. Primary outcome measure is time spent in platform quadrant (time (s)). Impaired learning for mice treated with PT +Abeta42 is expected at all time periods.

### Example 3: Learning Impairment in Mice Treated with Pertussis Toxin and Abeta42 Leads to Immunoglobulins in the Brain, Formation of FITC-labeled Plaques and Synaptic Decline

Histopathological alterations correlating with learning impairment are also investigated in mice treated with pertussis toxin and FITC-labeled Abeta42 (see Table 7 and are investigated for see Example 2). Pools of extracellular mouse IgGs around arterial vessels are expected in the mouse brains that were treated with PT and treated with PT and Abeta42. These pools of IgGs are not expected in the mice not treated with PT, as well as the mice in the group only injected with FITC-labeled Abeta42 alone. Similar observations of human IgGs were reported in human Alzheimer's disease brains [25,26].

Similarly, pools of FITC-labeling Abeta42 are expected around vessels like that of the mouse IgG. In addition, prominent vascular-derived, intracellular FITC-labeled Abeta42 is expected to be detectable in the neurons in the PT/Abeta42, treated mouse brains and in particular in the hippocampus and entorhinal cortex, areas prone to early pathology in Alzheimer's disease individuals. In contrast, no FITC-labeled Abeta42 should be detected in the other 3 groups of mice. However, neurons with high levels of FITC-labeling Abeta42 show signs of neurodegeneration as demonstrated by the condensed, pkynotic nuclei. In addition, FITC-positive amyloid plaques are expected to be detectable only in the 9-week treated mice providing evidence that over time, neurons endocytose vascular-derived, injected-FITC-labeling Abeta42 that die leaving the plaque.

The immunolabeling patterns of synaptophysin, an integral membrane glycoprotein in synaptic vesicles present in all synapses of neurons, should show normal punctate labeling in the mouse brains of the other 3 groups. Abnormal patterns (e.g., globular) of synaptophysin are expected to be observed in the molecular layers of the PT/Abeta42-treated mouse brains, and in some areas, less immunolabeling is detected. These observations suggest early morphological evidence of neuronal degeneration. Synaptic loss has been reported as an early phenomenon in Alzheimer's disease [60].

### Example 4: Use of A7R-specific Compounds to Reduce or Prevent Neuronal Death to Prevent Learning Impairment

A 9-week (and a 6-month learning study) is conducted to confirm that over time, vascular-derived Abeta will lead to learning impairment as further tested by the nesting test and the Morris water maze in addition to the established CognitiveWall test (see Example 1). The 9-week study will confirm that over time that vascular-derived Abeta not only leads to synaptic decline, neuronal degeneration, neuronal death, and amyloid plaque production, but also learning impairment through 3 behavioral models. In this study, several of the compounds from the in vitro study will be used in an established in vivo mouse model (see Example 2). After establishing a BBB leak via exogenous administration of pertussis toxin, FITC-labeled Abeta42 is injected with and without A7R compounds (see Table 1) through the tail vein to confirm the in vitro findings in vivo. Compounds will be administered on days with Abeta42 treatment (see Table 6) in an additional group of mice (Group 5).

Mice treated with A7R compounds and PT and FITC-labeled Abeta42 are expected to show decreased or no signs of behavioral impairment in all 3 behavioral tests (Congitive Wall, Morris water maze, and nesting) as compared to the same treated mice without the A7R compounds. Furthermore, histopathological evidence is expected to show decreased or no signs of neuronal degeneration in spite of pools of IgG and FITC-labeled Abeta around vessels.

The A7R agents are expected to prevent learning impairment over time in established mouse learning models of Alzheimer's disease. When the A7R compounds block toxic amounts of Abeta42 from entering neurons to save them from neuronal death, no learning impairment will be observed as compared with those mice not treated with A7R compounds exhibiting high levels of intraneuronal Abeta42, significant numbers of lysed neurons (=amyloid plaques), and learning impairment.

### REFERENCES

1. Acharyaa N K, Levina E C, Clifford P M, et al. Diabetes and hypercholesterolemia increase blood-brain barrier permeability and brain amyloid deposition: beneficial effects of the LpPLA2 inhibitor darapladib. J Alzheimers Dis. 2013;35:179-198.

2. Aisen P S. Review. The potential of anti-inflammatory drugs for the treatment ofAlzheimer's disease. Lancet Neurol 2002;1:27984.

3. Aisen P S, Davis K L, Berg J D, Schafer K, Campbell K, Thomas R G, et al. A randomized controlled trial of prednisone in Alzheimer's disease: Alzheimer's disease cooperative study. Neurology 2000;54:58893.

4. Aisen P S, Schafer K A, Grundman M, Pfeiffer E, Sano M, Davis K L, et al. Effects of rofecoxib or naproxen vs placebo on Alzheimer disease progression. JAMA 2003;289:281926.

5. Arendash G W, Sendstock G J, Sanberg P R, Kem W R. Improved learning and memory in aged rats with chromic administration of the nicotinic receptor agonist GTS-21. Brain Res. 1995;674:252-259.

6. Banerjee C, Nyengaard J R, Wevers A, et al. Cellular expression of α7 nicotinic acetylcholine receptor protein in the temporal cortex in Alzheimer's and Parkinson's disease: a stereological approach. Neurobiol Dis. 2000;7:666-672.

7. Bartus R T, Dean R L, Beer B, Lippa A S. The cholinergic hypothesis of geriatric memory dysfunction. Science. 1982;217:408-417.

8. Björkhem I, Meaney S. Brain cholesterol: long secret life behind the barrier. Arterioscler Thromb Vasc Biol. 2004;24:806-815.

9. Blanks J C, Schmidt S Y, Torigoe Y, Porrello K V, Hinton D R, Blanks R H. Retinal pathology in Alzheimer's disease II. Regional neuron loss and glial changes in GLC. Neurobiol Aging. 1996;17(3):385-395.

10. Blanks J C, Torigoe Y, Hinton D R, Blanks R H. Retinal pathology in Alzheimer's disease I. Ganglion cell loss in foveal/parafoveal retina. Neurobiol Aging. 1996;17(3):377-384.

11. Burghaus L, Schutz U, Krempel U, et al. Quantitative assessment of nicotinic acetylcholine receptor proteins in the cerebral cortex of Alzheimer patients. Brain Res Mol Brain Res. 2000;76:385-388.

12. Bowen D M, Smith C B, White P, Davison A N. Neurotransmitter-related enzymes and indices of hypoxia in senile dementia and other abiotrophies. Brain. 1976;99(3):459-496.

13. Breese C R, Adams C, Logel J, et al. Comparison of the regional expression of nicotinic acetylcholine receptor α7 mRNA and [¹²⁵I]-α-bungarotoxin binding in human postmortem brain. J Comp Neurol. 1997;387:385-398.

14. Cataldo A M, Nixon R A. Enzymatically active lyosomal proteases are associated with amyloid deposits in Alzheimer brain. Proc. Natl. Acad. Sci. US A 1990; 87; 3861-3865.

15. Chen D, Patrick J W. The a-bungarotoxin-binding nicotinic acetylcholine receptor from rat brain contains only the a7 subunit. J Biol Chem. 1997;272:24024-24029.

16. Clifford P M, Zarrabi S, Siu G, Kinsler K J, Kosciuk M C, D'Andrea M R, Nagele R G. Abeta peptides can enter the brain through a defective blood-brain barrier and bind selectively to neurons. Brain Res. 2007;1142:223-236.

17. Clifford P M, Siu G, Kosciuk M, Levin E, Venkataraman V, D'Andrea M R, Nagele R G. α7 nicotinic acetylcholine receptor expression by vascular smooth muscle cells facilitates the deposition of Aβ peptides and promotes cerebrovascular amyloid angiopathy. Brain Research. 2008; 1234:158-171.

18. Cogan D G. Visual disturbances with focal progressive dementing disease. Am J Ophthalmol. 1985;100:68-72.

19. Conejero-Goldberga C, Davies P, Ulloac L. Alpha7 nicotinic acetylcholine receptor: A link between inflammation and neurodegeneration. Neurosci Biobehav Rev. 2008;32(4):693-706.

20. Couturier S, Bertrand D, Matter J M, et al. A neuronal nicotinic acetylcholine receptor subunit (α7) is developmentally regulated and forms a homo-oligomeric channel blocked by a-BTX. Neuron. 1990;5:847-856.

21. Cronin-Golomb A, Sugiura R, Corkin S, Growdon J H. Incomplete achromatopsia in Alzheimer's disease. Neurobiol Aging. 1993;14(5):471-477.

22. Cunha-Vaz J G. Studies on the pathology of diabetic retinopathy. Diabetes. 1983;32(2):20-27

23. D'Andrea M R. Consequences of Intracellular Amyloid in Alzheimer's Disease. Elsevier Press, February, 2016.

24. D'Andrea M R. Bursting Neurons and Fading Memories: An Alternative Hypothesis of the Pathogenesis of Alzheimer's Disease. Elsevier Press, December, 2014.

25. D'Andrea M R. Evidence that the immunoglobulin-positive neurons in Alzheimer's disease are dying by the classical complement pathway. Am J Alzheimers Dis Other Demen. 2005;20(3):144-150.

26. D'Andrea M R. Evidence linking autoimmunity to neuronal cell death in Alzheimer's disease. Brain Res. 2003;982(1):19-30.

27. D'Andrea M R, Cole G M, Ard M D. The microglial phagocytic role with specific plaque types in the Alzheimer disease brain. Neurobiol Aging 2004;25:67583.

28. D'Andrea M R, Nagele R G. Morphologically distinct types of amyloid plaques point the way to a better understanding of Alzheimer's disease pathogenesis. Biotechnic and Histochemistry. 2010;85(2):133-47.

29. D'Andrea M R, Nagele R G. MAP-2 immunolabeling can distinguish diffuse from dense-core amyloid plaques in Alzheimer's disease brains. Biotechic & Histochemistry. 2002;77(2):95-103.

30. D'Andrea M R, Nagele R G. Targeting the alpha 7 nicotinic acetylcholine receptor to reduce amyloid accumulation in Alzheimer's disease pyramidal neurons. Current Pharmaceutical Design. 2006;12(6):677-684.

31. D'Andrea M R, Nagele R G, Gumula N A, Reiser P A, Polkovitch D A, Hertzog B M, Andrade-Gordon P. Lipofuscin and Abeta42 exhibit distinct distribution patterns in normal and Alzheimer's disease brains. Neuroscience Letters. 2002;323(1):45-49.

32. D'Andrea M R, Nagele R G, Wang H Y, Peterson P A, Lee D H S. Evidence that neurones accumulating amyloid can undergo lysis to form amyloid plaques in Alzheimer's disease. Histopathology. 2001;38:120-134.

33. D'Andrea M R, Reiser P A, Polkovitch D A, et al. The use of formic acid to embellish amyloid plaque detection in Alzheimer's disease tissues misguides key observations. Neurosci Lett. 2003;342(2):114-118.

34. D'Andrea M R, Reiser P A, Gumula N A, Hertzog B M, Andrade-Gordon P. Application of triple-label immunohistochemistry to characterize inflammation in Alzheimer's disease brains. Biotech Histochem. 2001;76(2):97-106.

35. Davies P, Maloney A J F. Selective loss of central cholinergic neurons in Alzheimer's disease. Lancet. 1976;2(8000):1403.

36. de La Torre J C. Vascular basis of Alzheimer's pathology. Ann N Y Acad Sci. 2002;977:196-215.

37. de la Torre J C. Is Alzheimer's disease a neurodegenerative or a vascular disorder? Data, dogma, and dialectics. Lancet Neurol. 2004;3(5):270.

38. Di Paolo G, Kim T W. Linking lipids to Alzheimer's disease: cholesterol and beyond. Nat Rev Neurosci. 2011;12(5):284-96.

39. Deutsch S I, Burket J A, Urbano M R, Benson A D. The a7 nicotinic acetylcholine receptor: A mediator of pathogenesis and therapeutic target in autism spectrum disorders and Down syndrome. Biochemical Pharmacology. 2015;97(4):363-377.

40. DeWitt D A, Perry G, Cohen M, Doller C, Silver J. Astrocytes regulate microglial phagocytosis of senile plaque cores of Alzheimer's disease. Exp. Neural. 1998;149:329-340.

41. Drachman D A, Leavitt J. Human memory and the cholinergic system. Arch Neurol. 1974;30:113-121.

42. Dowson J H. Neuronal lipopigment: a marker for cognitive impairment and long-term effects of psychotrophic drugs. Br J Psychiatry. 1989;155:1-11.

43. Dowson J H, Mountjoy C Q, Cairns M R, Wilton-Cox H. Changes in intraneuronal lipopigment in Alzheimer's disease. Neurobiol Aging. 1992;13:493-500.

44. Dowson J H, Mountjoy C Q, Cairns M R, Wilton-Cox H. Alzheimer's disease: distribution of changes in intraneuronal lipopigment in the frontal cortex. Dementia. 1995;6:334-342.

45. Eikelenboom P, van Gool W A. Review. Neuroinflammatory perspectives on the two faces of Alzheimer's disease. J Neural Trans 2004;111(3):28194.

46. Emmerling M R, Watson M D, Raby C A, Spiegel K. Review. The role of complement in Alzheimer's disease pathology. Biochimica et Biophysica Acta 2000;1502:15871.

47. Ennis S R, Betz A L. Sucrose permeability of the blood-retinal and blood-brain barriers. Invest Ophthalmol Vis Sci. 1986;27:1095-1102.

48. Eriksen J L, Sagi S A, Smith T E, Weggen S, Das P, McLendon D C, et al. NS AIDs and enantiomers of flurbiprofen target gamma-secretase and lower Abeta42 in vivo. J Clin Invest 2003;112:4409.

49. Farlow M R, Cummings J L. Effective pharmacologic management of Alzheimer's Disease. Am J Med. 2007;120(5):388-97.

50. Feldman H H, et al. Randomized controlled trial of atorvastatin in mild to moderate Alzheimer disease: LEADe. Neurology. 2010;74(12):956-64.

51. Francis P T, Patmer A M, Snape M, Wilcock G K. The cholinergic hypothesis of Alzheimer's disease: a review of progress. J Neurol Neurosurg Psychiatry. 1999;66:137-147.

52. Garbuzova-Davis S, Haller E, Williams S N, et al. Compromised blood-brain barrier competence in remote brain areas in ischemic stroke rats at chronic stage. J Comp Neurol. 2014;522(13):3120-3137.

53. Geifman N, Brinton R D, Kennedy R E, Schneider L S, Butte A J. Evidence for benefit of statins to modify cognitive decline and risk in Alzheimer's disease. Alzheimer's Research & Therapy. 2017;9:10.

54. Ginsberg S D, Galvin J E, Chiu T S, Lee V, Masliah E, Trojanowski J O. RNA sequestration to pathological lesions of neurodegenerative diseases. Acta Neuropathol. 1998;96:487-494.

55. Giulian D, Woodward J, Young D G, Krebs J F, Lachman L B. Interleukin-1 injected into mammalian brain stimulates astrogliosis and neovascularization. J. Neurosci. 1988;8:2485-2490.

56. Guan Z Z, Zhang X, Ravid R, Nordberg A. Decreased protein levels of nicotinic receptor subunits in the hippocampus and temporal cortex of patients with Alzheimer's disease. J Neurochem. 2000;74:237-243.

57. Guntert A, Dobeli H, Bohrmann B. High sensitivity analysis of amyloid beta peptide composition in amyloid deposits from human and PS2APP mouse brain. Neuroscience 2006;143(2):46175.

58. Haag M D, et al. Statins are associated with a reduced risk of Alzheimer disease regardless of lipophilicity. The Rotterdam Study. J Neurol Neurosurg Psychiatry. 2009;80(1):13-7.

59. Hartmann T. Role of amyloid precursor protein, amyloid-beta and gamma-secretase in cholesterol maintenance. Neurodegener Dis. 2006;3:305-311.

60. Heinonen O, Soininen H, Sorvari H, Kosunen O, Paljarvi L, Koivisto E, Riekkinen P J. Loss of synaptophysin-like immunoreactivity in the hippocampal formation is an early phenomenon in Alzheimer's disease. Neuroscience. 1995;64:2,375-384.

61. Hellstrom-Lindahl E, Mousavi M, Zhang X, Ravid R, Nordberg A. Regional distribution of nicotinic receptor subunit mRNAs in human brain: comparison between Alzheimer and normal brain. Mol Brain Res. 1999;66:94-103.

62. Herzig M C, Winkler D T, Burgermeister P, et al. Abeta is targeted to the vasculature in a mouse model of hereditary cerebral hemorrhage with amyloidosis. Nat Neurosci. 2004;7(9):954-960.

63. http://with the extension researchfeatures.com/2017/02/28/down-syndrome-accelerates-alzheimers-disease-onset/of the world wide web

64. https://with the extension alzheimersnewstoday.com/intepirdine-for-alzheimers-disease/of the world wide web

65. https://with the extension alz.org/facts/of the world wide web

66. https://with the extension alzforum.org/news/research-news/phase-3-solanezumab-trials-fail-there-silver-lining of the world wide web

67. https://with the extension alzforum.org/news/research-news/end-expedition-solanezumab-results-published of the world wide web

68. https://with the extension -alz.org/dementia/mild-cognitive-impairment-mci.asp of the world wide web

69. http://with the extension bmj.com/content/349/bmj.g5596 of the world wide web

70. https://with the extension businesswire.com/news/home/20180213006582/en/Merck-Announces-Discontinuation-APECS-Study-Evaluating-Verubecestat of the world wide web

71. https://with the extension fiercebiotech.com/biotech/merck-bacel-drug-fails-prodromal-alzheimer-s-phase-3 of the world wide web

72. https://with the extension fiercebiotech.com/r-d/why-did-roche-kill-off-its-alzheimer-s-program-for-bace-inhibitor-rg7129 of the world wide web

73. https://with the extension emedicinehealth.com/alzheimers_disease_in down_syndrome/page2_em.htm #what_is_the_link_between_down syndrome_and_alzheimers disease of the world wide web

74. https://with the extension emedicine.medscape.com/article/1136117-overview of the world wide web

75. https://with the extension en.wikipedia.org/wiki/Down_syndrome of the world wide web-

76. http://with the extension everydayhealth.com/alzheimers/alzheimers-risk-factors.aspx of the world wide web

77. http://with the extension forbes.com/sites/matthewherper/2012/08/08/how-a-failed-alzheimers-drugillustrates-the-drug-industrys-gambling-problem/of the world wide web

78. https://with the extension ft.com/content/e77fe5a2-01e8-11e8-9e12-af73e8db3c71?sharetype=share of the world wide web

79. https://with the extension my.clevelandclinic.org/health/articles/alzheimers-disease-and-down-syndrome?view=print of the world wide web

80. https://with the extension ndss.org/about-down-syndrome/down-syndrome/of the world wide web

81. https://with the extension nia.nih.gov/health/alzheimers-disease-people-down-syndrome of the world wide web

82. https://with the extension npr.org/sections/thetwo-way/2018/01/08/576443442/pfizer-halts-research-efforts-into-alzheimers-and-parkinsons-treatments of the world wide web

83. https://with the extension wsj.com/articles/axovant-will-no-longer-develop-lead-dementia-drug-1515430108 of the world wide web

84. http://with the extension report.nih.gov/categorical_spending.aspx of the world wide web

85. http://with the extension researchfeatures.com/2017/02/28/down-syndrome-accelerates-alzheimers-disease-onset/of the world wide web

86. https://with the extension patents.google.com/patent/US20030013699A1/en?oq=%E2%80%A2U.S.+Patent+Application+No.+2003%2f0013699+of the world wide web

87. https://with the extension patents.google.com/patent/US20040235850A1/en?q=varenicline&q=alzheimer r&num=50 of the world wide web

88. https://with the extension patents.google.com/patent/US20080286340A1/en?q=nicotine&q=alzheimer%2 7s+disease&num=50 of the world wide web

89. https://with the extension patents.google.com/patent/U.S. Pat. No. 5,278,176A/en?q=nicotine&q=alzheimer%27s+disease&num=50 of the world wide web

90. https://with the extension patents.google.com/patent/U.S. Pat. No. 5,069,904A/en?q=nicotine&q=alzheimer%27s+disease&num=50 of the world wide web

91. https://with the extension patents.google.com/patent/U.S. Pat. No. 6,416,735B1/en?q=methyllycaconitine;&q=alzheimer%27s+disease&num=50 of the world wide web

92. https://with the extension patents.google.com/patent/WO2002020016A1/en?q=methyllycaconitine;&q=alzheimer%27s+disease&num=50 of the world wide web

93. https://with the extension patents.google.com/patent/U.S. Pat. No. 5,977,144A/en?q=methods&q=use&q=composition &q=benzylidene-&q=cinnamylidene-anabaseines&q=treat&q=Alzheimer%27s+disease&oq=claim+methods+of+use+and+composition+for+benzylidene-+and+cinnamylidene-anabaseines+ to+treat+Alzheimer%27s+disease+of the world wide web

94. https://with the extension patents.google.com/patent/U.S. Pat. No. 6,211,194B1/en?oq=patent+U.S. Pat. No. 6,211,194B1 of the world wide web

95. https://with the extension patents.google.com/patent/US20070060588A1/en?oq=patent+US20070060588A 1 of the world wide web

96. https://with the extension patents.google.com/patent/U.S. Pat. No. 8,592,458 of the world wide web

97. http://with the extension scitechconnect.elsevier.com/alzheimers-disease-do-the-eyes-really-have-it/of the world wide web

98. https://with the extension npr.org/sections/health-shots/2016/08/31/491941518/test-of-experimental-alzheimers-drug-finds-progress-against-brain-plaques of the world wide web

99. https://with the extension statnews.com/2018/01/08/alzheimers-axovant-intepirdine/of the world wide web

100. https://with the extension webmd.com/heart-disease/common-medicine-heart-disease-patients#1 of the world wide web

101. http://with the extension who.int/en/news-room/fact-sheets/detail/dementia\ of the world wide web

10I https://with the extension wsj.com/articles/axovant-says-experimental-drug-failed-trials-in-treating-alzheimers-disease-1506429031 of the world wide web

103. https://with the extension youtube.com/watch?v=_NTaGjQow1c of the world wide web

104. Imbimbo B P, Solfrizzi V, Panza F. Are NSAIDs useful to treat Alzheimer's disease or mild cognitive impairment? Frontiers in Aging Neuroscience. 2010:2(19):1-14.

105. Jacobsen E J, Myers J K, Walker D P, et al. Azabicyclic compounds for the treatment of disease. U.S. Pat. No. 7,001,900B2. Feb. 21 2006.

106. Jarvik G P, et al. Interactions of apolipoprotein E genotype, total cholesterol level, age, and sex in prediction of Alzheimer's disease: a case-control study. Neurology. 1995;45(6):1092-6.

107. Jiang X, Guo M, Su J, et al. Simvastatin blocks blood-brain barrier disruptions induced by elevated cholesterol both in vivo and in vitro. Int J Alzheimers Dis. 2012;2012:109325. doi: 10.1155/2012/109325.

108. Jick H, et al. Statins and the risk of dementia. Lancet. 2000;356(9242):1627-31.

109. Kanekiyo T, Xu H, Bu G. ApoE and Abeta in Alzheimer's disease: accidental encounters or partners? Neuron. 2014;81(4):740-54.

110. Katz B, Rimmer S. Ophthalmologic manifestations of Alzheimer's disease. Sury Ophthalmol. 1989;34:31-43.

111. Kem W R. The brain alpha7 nicotinic receptor may be an important therapeutic target for the treatment of Alzheimer's disease: studies with DMXBA (GTS-21). Behavioral Brain Res. 2000;113:169-181.

112. Kivipelto M, Helkala E L, Lasskso M P, et al. Midlife vascular risk factors and Alzheimer's disease in later life: longitudinal, population based study. BMJ. 2001;322(7300):1447-1451.

113 Knopman D S, Petersen R C. Mild cognitive impairment and mild dementia: A clinical perspective. May Clin Proc. 2014;89(10):1452-1459.

114. Krogsaa B, Lund-Andersen H, Parving H H, Bjaeldager P. The blood-retinal barrier permeability in essential hypertension. Acta Ophthalmol. 1983;16(4):541-544.

115. Krstic D, Knuesel I. Deciphering the mechanism underlying late-onset Alzheimer disease. Nat Rev Neurol. 2013;9:25-34.

116. Kugler S, Bocker K, Heusipp G, et a;. Pertussis toxin transiently affects barrier integrity, organelle organization and transmigration of monocytes in a human brain microvascular endothelial cell barrier model. Cellular Microbiology. 2007;9(3):619-632.

117. Lee D H S, D'Andrea M R, Plata-Salaman C R, Wang H-Y. Decreased α7 nicotinic acetylcholine receptor proteins in sporadic Alzheimer's disease brains. Alzheimer's Letters. 2000;3(4):217-220.

118. Lee S, Jadhav V, Lekic T, et al. Simvastatin treatment in surgically induced brain injury in rats. Acta Neurochir Suppl. 2008;102:401-404.

119. Lim G P, Yang F, Chu T, Chen P, Beech W, Teter B, et al. Ibuprofen suppresses plaquepathology and inflammation in a mouse model for Alzheimer's disease. J Neurosci2000;20:5709 14.

120. Lindenberg R, Haymaker W. Tissue reactions in the gray matter of the central nervous system, In W. Haymaker and R. Adams (Eds.), Histology and Histopathology of the Nervous System, Vol. 1, Charles C. Thomas, Springfield, Ill., 1982 pp. 973-1275.

121. Li C, Zhao R, Gao K, Wei Z, Yin M Y, Lau L T, et al. Review. Astrocytes: implications for neuroinflammatory pathogenesis of Alzheimer's disease. Curr Alzheimer Res 2011;8(1):6780.

122. Liu L, Chan C. Review. The role of inflammasome in Alzheimer's disease. Ageing Red Rev 2014;615.

123. Liu Q, Xie X, Emadi S, Sierks M R, Wu J. A novel nicotinic mechanism underlies β-amyloid-induced neurotoxicity. Neuropharmacology. 2015;97:457-463.

124. Lucas H R, Rifkind J M. Considering the vascular hypothesis of Alzheimer's disease: effect of copper associated amyloid on red blood cells. Adv Exp Med Biol. 2013;765:131-138.

125. Lund-Andersen H. Mechanisms for monitoring changes in retinal status following therapeutic intervention in diabetic retinopathy. Sury Ophthalmol. 2002;47(52):S270-5277.

126. Luster A D. Chemokines-chemotactic cytokines that mediate inflammation. N Engl J Med 1998;338:43645.

127. McGeer P L, Schulzer M, McGeer E G. Review. Arthritis and anti-inflammatory agents as possible protective factors for Alzheimer's disease: a review of 17 epidemiologic studies. Neurology 1996;47:42532.

128. McGeer E G, McGeer P L. Review. The importance of inflammatory mechanisms in Alzheimer disease. Exp Gerontol 1998;33:3718.

129. McGeer P L, McGeer E G. Review. Inflammation of the brain in Alzheimer's disease: implications for therapy. J Leukocyte Biol 1999;65:40915.

130. McGehee D S, Heath M J, Gelber S, Devay P, Role L W. Nicotine enhancement of fast excitatory synaptic transmission in CNS by presynaptic receptors. Science. 1995;269:1692-1696.

131. McGuinness B, et al. Statins for the treatment of dementia. Cochrane Database Syst Rev. 2014;7:CD007514.

132. Mihalak K B, Carroll F I, Leutje C W. Varenicline is a partial agonist at α4β2 and a full agonist at α7 neuronal nicotinic receptor. Mol Pharm. 2006;70(3):801-805.

133. Mohandas E, Rajmohan V, Raghunath B. Neurobiology of Alzheimer's disease. Indian J Psychiatry. 2009;51(1):55-61.

134. Mooradian A D, Haas M J, Batejko O, Hovsepyan M, Feman S S. Statins ameliorate endothelial barrier permeability changes in the cerebral tissue of streptozotocin-induced diabetic rats. Diabetes. 2005;54(10):2977-2982.

135. Moore A H, O'Banion M K. Neuroinflammation and anti-inflammatory therapy for Alzheimer's disease. Adv Drug Deliv Rev. 2002;54:1627-1656.

136. Mortel K F, Wood S, Pavol M A, Meyer J S, Rexer J L. Analysis of familial and individual risk factors among patients with ischemic vascular dementia and Alzheimer's disease. Angiology. 1993;44(8):599-605.

137. Nagaraja T N, Knight R A, Croxen R L, Konda K P, Fenstermacher J D. Acute neurovascular unit protection by simvastatin in transient cerebral ischemia. Neurol Res. 2006;28(8):826-830.

138. Nagele, R G, D'Andrea M R, Lee H, Venkataraman V, Wang H Y. Astrocytes accumulate Abeta42 and give rise to astrocytic amyloid plaques in Alzheimer disease brains. Brain Research. 2003;971:197-209.

139. Nagele R G, D'Andrea M R, Anderson W J, Wang H-Y. Intraneuronal accumulation of β-amyloid 1-42 is mediated by the α7 nicotinic acetylcholine receptor in Alzheimer's disease. Neuroscience. 2002;110(2):199-211.

140. Nagga K, Wattmo C, Zhang Y, Wahland L-O, Palmqvist S. Cerebral inflammation is an underlying mechanism of early death in Alzheimer's disease: a 13-year cause-specific multivariate mortality study. Alzheimers Res Ther. 2014;6:41.

141. Nilsson L, Nordberg A, Hardy J A, Wester P, Winblad B. Physostigmine restores [3H]-acetylcholine efflux from Alzheimer brain slices to normal level. J Neural Transm. 1986;67:275-285.

142. Norberg A. Human nicotinic receptors-their role in aging and dementia. Neurochem Int. 1994;25:93-97.

143. Olincy A, Stevens K E. Treating schizophrenia symptoms with an alpha7 nicotinic agonist, from mice to men. Biochem Pharm. 2007;74:1192-1201.

144. Paterson D, Norber A. Neuronal nicotinic receptors in the human brain. Prog Neurobiol. 2000;6:75-111.

145. Perry E K, Court J A, Johnson M, Piggott M A, Perry R H. Autoradiographic distribution of [³H]nicotine binding in human cortex: Relative abundance in subicular complex. J Chem Neuroanat. 1992;5:399-405.

146. Perry E K, Gibson P H, Blessed G, Perry R H, Tomlinson B E. Neurotransmitter enzyme abnormalities in senile dementia. Choline acetyltransferase and glutamic acid decarboxlyase activities in necropsy brain tissue. J Neurol Sci. 1977;34:247-265.

147. Peng J H, Lucero L, Fryer J, et al. Inducible, heterologous expression of human α7-nicotinic acetylcholine receptors in a native nicotinic receptor-null human clonal line. Brain Res. 1999;825:172-179.

148. Petanceska S S, et al. Statin therapy for Alzheimer's disease: will it work? J Mol Neurosci. 2002;19(1-2):155-61.

149. Petanceska S S, et al. Changes in apolipoprotein E expression in response to dietary and pharmacological modulation of cholesterol. J Mol Neurosci. 2003;20(3):395-406.

150. Quik M, Philie J, Choremis J. Modulation of alpha7 nicotinic receptor-mediated calcium influx by nicotinic agonist. Mol Pharmacol. 1997;51(3):499-506.

151. Refolo L M, et al. A cholesterol-lowering drug reduces beta-amyloid pathology in a transgenic mouse model of Alzheimer's disease. Neurobiol Dis. 2001;8(5):890-9.

152. Rockwood K, et al. Use of lipid-lowering agents, indication bias, and the risk of dementia in community-dwelling elderly people. Arch Neurol. 2002;59(2):223-7.

153. Rogers J, Morrison J H. Quantitative morphology and regional and laminar distributions of senile plaques in Alzheimer's disease. J. Neurosci. 1985;5:2801-2808.

154. Rojo L E, Fernandez J A, Maccioni A A, Jimenez J M, Maccioni R B. Review. Neuroinflammation: implications for the pathogenesis and molecular diagnosis of Alzheimer's disease. Arch Med Res 2008;39(1):116.

155. Rylett R j, Ball M J, Colhuon E H. Evidence for high affinity choline transport in synaptosomes prepared from hippocampus and neocortex of patients with Alzheimer's disease. Brain Res. 1983;289:169-175.

156. Sano M, et al. A randomized, double-blind, placebo-controlled trial of simvastatin to treat Alzheimer disease. Neurology. 2011;77(6):556-63.

157. Sastre M, Klockgether T, Heneka M T. Review. Contribution processes to Alzheimer's disease: molecular mechanisms. Int J Neurosci 2006;24:16776.

158. Schroder H, Wevers A. Nicotinic acetylcholine receptors in Alzheimer's disease. Alzheimer's Dis Rev. 1998;3: 20-27.

159. Seguela P, Wadiche J, Dineley-Miller K, et al. Molecular cloning, functional properties, and distribution of rat brain alpha 7: a nicotinic cation channel highly permeable to calcium. J Neurosci. 1993;13(2):596-604.

160. Shoham S, Ebstein R P. The distribution of (3-amyloid precursor protein in rat cortex after systemic kainite-induced seizures. Exp Neurol 1997;147:36176.

161. Simons M, et al. Cholesterol and Alzheimer's disease: is there a link? Neurology. 2001;57(6):1089-93.

162. Simons M, et al. Treatment with simvastatin in normocholesterolemic patients with Alzheimer's disease: a 26-week randomized, placebo-controlled, double-blind trial. Ann Neurol. 2002;52(3):346-50.

163. Skoog I. Risk factors for vascular dementia: a review. Dementia. 1994;5:137-144.

164. Sparks D L, et al. Link between heart disease, cholesterol, and Alzheimer's disease: a review. Microsc Res Tech. 2000;50(4):287-90.

165. Su G C, Arendash G W, Kalaria R N, Bjugstad K B, Mullan M. Intravascular infusions of soluble beta-amyloid compromise the blood-brain barrier, activate CNS glial cells and induce peripheral hemorrhage. Brain Res. 1999;818(1):105-117.

166. Szekely CA, Zandi P P. Non-steroidal anti-inflammatory drugs and Alzheimer's disease: the epidemiological evidence. CNS Neurol Disord Drug Targets. 2010;9(2):132-139.

167. Tatton W T, Chen D, Charmers-Redman R, Wheeler L, Nixon R, Tatton N. Hypothesis of a common basis for neuroprotection in glaucoma and Alzheimer's disease: anti-apoptosis by alpha-2-adrenergic receptor activation. Sury Ophthalmol. 2003;48(1):S25-S37.

168. Tuppo E E, Arias H R. Review. The role of inflammation in Alzheimer's disease. Int J Biochem Cell Bio 2005;37:289305.

169. van Vliet E A, da Costa Araujo S, Redeker S, van Schaik R, Aronica E, Gorter J A. Blood-brain barrier leakage may lead to progression of temporal lobe epilepsy. Brain. 2007;1302:521-534.

170. Vinores S A, Kuchle M, Mahlow J, Chiu C, Green W R, Campochiaro P A. Blood-ocular barrier breakdown in eyes with ocular melanoma. Am J Pathol. 1995;147(5):1289-1297.

171. Vinores S A. Localization of blood-retinal barrier breakdown in human pathologic specimens by immunohistochemical staining for albumin. Lab Invest. 1990;62(6):742-750.

172. Wang H Y, Lee D H, D'Andrea M R, Peterson O A, Shank R P, Reitz A B. Beta-Amyloid(1-42) binds to alpha7 nicotinic receptor with high affinity. Implications for Alzheimer's disease pathology. J Bio Chem. 2000;275(8):5626-5628.

173. Wang H Y, Lee D H, Davis C B, Shank R P. Amyloid peptide Abeta(1-42) binds selectively and with picomolar affinity to alpha7 nicotinic acetylcholine receptors. J Neurochem. 2000;75(3):1155-1162.

174. Wang H Y, D'Andrea M R, Nagele R G. Cerebellar diffuse amyloid plaques are derived from dendritic Abeta42 accumulations in Purkinje cells. Neurobiology of Aging. 2002;23(2):213-223.

175. Wang S. Key to detecting Alzheimer's early could be in the eye. The Wall Street Journal. Jul. 13, 2014.

176. Weggen S, Eriksen J L, Das P, Sagi S A, Wang R, Pietrzik C U, et al. A subset of NS AIDs lower amyloidogenic Abeta42 independently of cyclooxygenase activity. Nature 2001;414:21216.

177. Wellington C L. Cholesterol at the crossroads: Alzheimer's disease and lipid metabolism. Clin Genet. 2004;66:1-16.

178. Wevers A, Burghaus L, Moser N, et al. Expression of nicotinic acetylcholine receptors in Alzheimer's disease: postmortem investigations and experimental approaches. Behav Brain Res. 2000;113:207-215.1.

179. Weyer S W, Klevanski M, Delekate A, et al. APP and APLP2 are essential at PNC and CNS synapses for transmission, spatial learning and LTP. The EMBO Journal. 2011;30:2266-2280.

180. Whitehouse P J, Kalaria R N. Nicotinic receptors and neurodegenerative dementing diseases: basic research and clinical implications. Alzheimer Dis Assoc Disord. 1995;9:3-5.

181. Whitehouse P J, Price D L, Struble R G, Clarke A, Coyle J, Delong M. Alzheimer's disease and senile dementia: loss of neurons in basal forebrain. Science. 1982;215:1237-1239.

182. Winslow B T, Onysko M K, Stob C M, Hazlewood K A. Treatment of Alzheimer Disease. Am Fam Physician. 2011;83(12):1403-1412.

183. Wolozin B, et al. Decreased prevalence of Alzheimer disease associated with 3-hydroxy-3-methyglutaryl coenzyme A reductase inhibitors. Arch Neurol. 2000;57(10):1439-43.

184. Wong T Y. Is retinal photography useful in the measurement of stroke risk? Lancet. 2004;3:179.

185. www.alz.org (accessed June, 2014 of the world wide web)

186. Xia M Q, Hyman B T. Review. Chemokines/chemokine receptors in the central nervous system and Alzheimer's disease. J Neurovirol 1999;5:3241.

187. Yao J, et al. Aging, gender and APOE isotype modulate metabolism of Alzheimer's Abeta peptides and F-isoprostanes in the absence of detectable amyloid deposits. J Neurochem. 2004;90(4):1011-8.

188. Yang T, Xiao T, Sun Q, Wang K. The current agonists and positive allosteric modulators of alpha7 nAChR for CNS indications in clinical trials. Acta Pharmaceutica Sinica B. 2017;7(6):611-622.

189. Yin D. Biochemical basis of lipofuscin, ceroid, and age pigment-like fluorophores. Free Radic Biol Med. 1996;21:871-888.

190. Zhang Z G, Zhang L, Jiang Q, et al. VEGF enhances angiogenesis and promotes blood-brain barrier leakage in the ischemic brain. J Clin Invest. 2000;106(7):829-838.

1. A method for blocking or reducing toxic accumulation of amyloid in cells, with said method comprising administering one or more specific A7R binding agents to the cells.

2. The method of claim 1 wherein the cells are neurons, smooth muscle cells, or endothelial cells.

3. A method for preventing, inhibiting, or delaying onset of Alzheimer's disease and other forms of dementia and mild cognitive impairment (MCI) said method comprising administering to a subject one or more specific A7R binding agents so that toxic accumulation of amyloid in cells of the subject is blocked or reduced.

4. The method of claim 3 wherein the A7R binding agent is administered to a subject prior to the onset of Alzheimer's disease.

5. The method of claim 4 wherein the subject has Dementia, a leaky BBB, and/or MCI or is at risk of developing MCI.

6. The method of claim 3 wherein the A7R binding agent is administered to a subject at risk for developing Alzheimer's disease.

7. The method of claim 6 wherein the subject has Down syndrome, diabetes, high blood pressure, vascular disease, a genetic predisposition to Alzheimer's disease and/or serum markers of neuronal debris.

8. The method of any of claim 3 further comprising administering to the subject one or more agents for cardiovascular pathology with minimizes BBB leakage and/or one or more agents which reduces neuroinflammation in the brain activated from neuronal death.

9. The method of claim 8 wherein the agent for cardiovascular pathology which minimizes BBB leakage is an inhibitor of lipoprotein-associated phospholipase-A2, a statin, VEGF, or other agents that promote BBB function.

10. The method of claim 8 wherein the agent which reduces neuroinflammation is steroidal or nonsteroidal anti-inflammatory drug.

11. A combination therapy for the prevention, inhibition, or delay of onset of Alzheimer's disease or other forms of dementia and MCI comprising one or more A7R binding agents and one or more agents for cardiovascular pathology which minimizes BBB leakage and/or one or more agents which reduces neuroinflammation in the brain activated from neuronal death.

12. The method of claim 3 further comprising identifying the individual to be at risk for developing Alzheimer's disease by assessing BBB health in the individual.

13. The method of claim 12 wherein BBB health is assessed by BRB health and/or detection of beta-amyloid or any vascular protein/agent that leaked into the eye.

14. The combination therapy of claim 11 wherein the agent for cardiovascular pathology which minimizes BBB leakage is an inhibitor of lipoprotein-associated phospholipase-A2, a statin, VEGF, or other agents that promote BBB function.

15. The combination therapy of claim 11 wherein the agent which reduces neuroinflammation is steroidal or nonsteroidal anti-inflammatory drug.
